(19) United States
(12) Patent Application Publication
(10) Pub. No.: US 2024/0009265 A1
(43) Pub. Date: Jan. 11, 2024

(54) METHODS OF TREATMENT COMPRISING STABLE REDUCED GLUTATHIONE

(71) Applicant: Blue Sky Innovation, LLC, Pleasant Grove, UT (US)

(72) Inventors: Danny C. Purser, Provo, UT (US); Stephen N. Pitcher, Alpine, UT (US)

(73) Assignee: Blue Sky Innovation, LLC, Pleasant Grove, UT (US)

(21) Appl. No.: 17/922,487

(22) PCT Filed: Apr. 30, 2021
(86) PCT No.: PCT/US2021/030326

§ 371 Date: Oct. 31, 2022

Related U.S. Application Data
(60) Provisional application No. 63/018,480, filed on Apr. 30, 2020.

(51) Int. Cl. A61K 38/06; A61K 9/00; A61P 17/02; A61K 31/545; A61K 9/127

(52) U.S. Cl. CPC A61K 38/063; A61K 9/0014; A61P 17/02; A61K 31/545; A61K 9/127

(57) ABSTRACT

The present disclosure is drawn to a method of treating a subject having a condition responsive to treatment with reduced glutathione (rGSH), comprising: administering a therapeutically effective amount of a substantially α-D-glucopyranoside-free stabilized rGSH composition to the subject.

### PRIORITY DATA

This application is a 371 U.S. Nationalization of International Patent Cooperation Treaty Application No. PCT/US2021/030326, filed Apr. 30, 2021, which claims the benefit of U.S. Provisional Application Ser. No. 63/018,480, filed Apr. 30, 2020, each of which is incorporated herein by reference in their entirety.

### TECHNOLOGY FIELD

The present disclosure relates to compositions and methods of treatment using reduced glutathione. Accordingly, this disclosure involves the fields of chemistry, pharmaceutical sciences, medicine and other health sciences.

### BACKGROUND OF THE DISCLOSURE

Glutathione is a tripeptide comprised of three amino acids (cysteine, glutamic acid, and glycine) that can prevent damage to cellular components caused by reactive oxygen species (ROS) such as free radicals. Glutathione can exist in a reduced state (rGSH) and an oxidized state (GSSG). In healthy cells, more than 90% of the glutathione can be in the reduced state. Reduced glutathione plays various roles including in antioxidation, in detoxification, in thiol-exchange reactions, in metabolism, as an enzyme cofactor, and in leukotriene synthesis.

Bioavailability of exogenously administered rGSH can be poor because of reactions with reactive oxygen species during product storage and administration. Accordingly, in view of the potential therapeutic benefits offered by rGSH, stabile compositions and methods of administration that place a maximum amount of rGSH at target treatment situs continue to be sought.

### SUMMARY OF THE DISCLOSURE

The present disclosure is drawn to methods of treating a subject having a condition responsive to treatment with reduced glutathione (rGSH). Such methods can comprise administering a therapeutically effective amount of a substantially α-D-glucopyranoside-free stabilized rGSH composition to the subject.

In one embodiment, a method of treating a skin disease or condition is provided. Such a method can comprise administering a therapeutically effective amount of a substantially α-D-glucopyranoside-free stabilized rGSH composition to the skin of the subject. The composition can be a in a topical dosage form that can comprise a stabilizing carrier, such as deoxygenated water. The composition can further comprise an additional active agent, such as an antibiotic or an antioxidant. In some embodiments, for example, when the skin condition is a burn, the rGSH can be present in the composition in an amount of from about 50 mg to about 1500 mg.

In another embodiment, a method of treating a neurological disease or condition is provided. In one aspect, such a method can comprise administering a therapeutically effective amount of a substantially α-D-glucopyranoside-free stabilized rGSH composition to the subject. In some embodiments, the composition administered can be a topical dosage form that can comprise a stabilizing carrier such as deoxygenated water. The composition can further comprise an additional active agent, such as an antioxidant or an antiviral. In yet further aspect, for example, when the neurological condition is symptomized by a motor effect, or by pain, the rGSH can be present in the composition in an amount of from about 50 mg to about 1500 mg.

In another embodiment, a method of treating an inflammatory disease or condition is provided. In one aspect, such a method can comprise administering a therapeutically effective amount of a substantially α-D-glucopyranoside-free stabilized rGSH composition to the subject. The composition administered can be in a topical dosage form or an oral dosage form, or a combination thereof that can comprise a stabilizing carrier, such as deoxygenated water. The composition can further comprise an additional active agent, such as an antioxidant. In some aspects, for example when the inflammatory condition is fibromyalgia, the rGSH can be present in the composition in an amount of from about 50 mg to about 1500 mg.

In another embodiment, a method of treating a viral disease or condition is provided. In one aspect, such a method can comprise administering a therapeutically effective amount of a substantially α-D-glucopyranoside-free stabilized rGSH composition to the subject. The composition administered can be in a transmucosal dosage form or an oral dosage form that can comprise a stabilizing carrier, such as deoxygenated water. The composition can further comprise an additional active agent, such as an antiviral. In some aspects, for example, when the viral condition is post-herpetic neuralgia (PHN), the rGSH can be present in the composition in an amount of from about 50 mg to about 1500 mg.

In yet another embodiment, a method of treating a disease or condition responsive to treatment with an antioxidant is provided. In some aspects, such a method can comprise administering a therapeutically effective amount of a substantially α-D-glucopyranoside-free stabilized rGSH composition to the subject. The composition administered can be in an oral dosage form that can comprise a stabilizing carrier, such as deoxygenated water. In some aspects, the rGSH can be present in the composition in an amount of from about 50 mg to about 1500 mg.

In yet a further embodiment, a method of stimulating a subject's immune response is provided. In one aspect, such a method can comprise administering a therapeutically effective amount of a substantially α-D-glucopyranoside-free stabilized rGSH composition to the subject. The composition administered can be in an oral dosage form that can comprise a stabilizing carrier, such as deoxygenated water. In some aspects, the rGSH can be present in the composition in an amount of from about 50 mg to about 1500 mg.

### BRIEF DESCRIPTION OF THE DRAWINGS

For a fuller understanding of the nature and advantage of the present disclosure, reference is being made to the following detailed description of preferred embodiments and in connection with the accompanying drawings, in which:

FIG. 1a is a chart of total antioxidant function and immunidex in accordance with an example;

FIG. 1B is a chart of total antioxidant function and immunidex in accordance with an example;

FIG. 1c is a chart of total antioxidant function and immunidex in accordance with an example;

FIG. 1d is a chart of total antioxidant function and immunidex in accordance with an example;

FIG. 1e is a chart of total antioxidant function and immunidex in accordance with an example;

FIG. 1f is a chart of total antioxidant function and immunidex in accordance with an example;

FIG. 2 is a graph of the average immunidex based on age group in accordance with an example; and

FIG. 3 depicts a mechanism relating hyperglycemia and oxidative stress.

### DETAILED DESCRIPTION

Before disclosure embodiments are described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular examples or embodiments only and is not intended to be limiting. The same reference numerals in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of compositions, dosage forms, treatments, etc., to provide a thorough understanding of various disclosure embodiments. One skilled in the relevant art will recognize, however, that such detailed embodiments do not limit the overall inventive concepts articulated herein, but are merely representative thereof.

The present disclosure is drawn to a method of treating a subject having a condition responsive to treatment with reduced glutathione (rGSH). The method can comprise administering a therapeutically effective amount of a substantially α-D-glucopyranoside-free stabilized rGSH composition to the subject. The composition can be in a dosage form that can comprise a stabilizing carrier, such as deoxygenated water.

It should be noted that, the singular forms “a,” “an,” and, “the” include plural referents unless the context clearly dictates otherwise. Thus, for example, reference to “an excipient” includes reference to one or more of such excipients, and reference to “the carrier” includes reference to one or more of such carriers.

As is known in the art, the term “reduced glutathione” or “rGSH” generally refers to a compound having the structure:

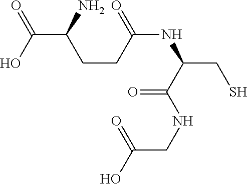 [SMILES: N[C@@H](CCC(=O)N[C@@H](CS)C(=O)NCC(=O)O)C(=O)O]

As used herein, the terms “oxidized glutathione” or “GSSG” or “glutathione disulfide” can be used interchangeably and refer to a compound having the structure:

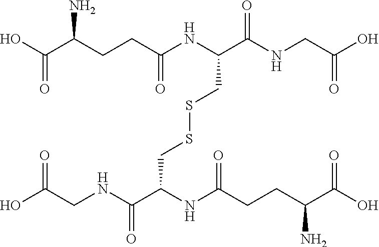 [SMILES: N[C@@H](CCC(=O)N[C@@H](CSSC[C@H](NC(=O)CC[C@H](N)C(=O)O)C(=O)NCC(=O)O)C(=O)NCC(=O)O)C(=O)O]

As used herein, the terms “treat,” “treatment,” or “treating” refers to administration of a therapeutic agent to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, the terms “therapeutic agent,” “active agent,” and the like can be used interchangeably and refer to agent that can have a beneficial or positive effect on a subject when administered to the subject in an appropriate or effective amount. In one aspect, the therapeutic or active agent can be an rGSH compound. The terms “additional active agent,” “supplemental active agent,” “secondary active agent,” and the like can be used interchangeably and refer to a compound, molecule, or material other than rGSH that has physiologic activity when administered to a subject in an effective amount.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients. Furthermore, the term “dosage form” can include one or more formulation(s) or composition(s) provided in a format for administration to a subject. For example, an “oral dosage form” can be suitable for administration to a subject's mouth. A “topical dosage form” can be suitable for administration to a subject's skin by rubbing, etc.

As used herein, the term “stabilizing agent” refers to a compound, molecule, or substance that has a stabilizing effect on rGSH or an additional active agent and increases the stability (e.g. reduces the degradation of rGSH into GSSG) while in a composition or dosage form.

As used herein, the term “fatty acid” refers to unionized carboxylic acids with a long aliphatic tail (chain), either saturated or unsaturated, conjugated or non-conjugated.

As used herein, the term “soluble” is a measure or characteristic of a substance or agent with regards to its ability to dissolve in a given solvent. The solubility of rGSH in a particular component of the composition refers to the amount of the rGSH dissolved to form a visibly clear solution at a specified temperature such as about 25° C. or about 37° C.

As used herein, the term “lipophilic,” refers to compounds that are not freely soluble in water; and the term “lipophilic surfactant” refers to surfactants that have HLB values of about 10 or less. Conversely, the term “hydrophilic” refers to compounds that are soluble in water; and the term “hydrophilic surfactant” refers to surfactants that have HLB values of more than about 10.

As used herein, the term “capsule fill” refers to the composition disposed in a capsule dosage form.

As used herein, a “subject” refers to an animal. In one aspect the animal may be a mammal. In another aspect, the mammal may be a human.

A used herein, a “responder” is a subject who responds positively to rGSH treatment or therapy. “Responder analysis” is the assessment of the effectiveness of rGSH therapy in a group of subjects deemed to get benefits of rGSH therapy.

As used herein, “group” or “group of subjects” refers to a collection of at least 3 human subjects who receive and respond to administration of the compositions disclosed herein, namely rGSH compositions. In one aspect, the group can include at least 20 or at least 50 subjects. In another aspect, the group can include at least 100 subjects.

The term “oral administration” represents any method of administration in which an active agent can be administered by swallowing, chewing, or sucking of the dosage form. Oral administration can be intended for enteral delivery of an active agent or transmucosal delivery of the active agent. In some embodiments, the composition of the current disclosures can be admixed with food or drink prior to being orally consumed.

As used herein, a “dosing regimen” or “regimen” such as an “initial dosing regimen” or “starting dose” or a “maintenance dosing regimen” refers to how, when, how much, and for how long a dose of the compositions of the present disclosure can be administered to a subject. For example, an initial or starting dose regimen for a subject may provide for a total daily dose of 600 mg administered in two divided doses at least 12 hours apart (e.g. once with breakfast and once with dinner) with meals repeated daily for days.

As used herein, “daily dose” refers to the amount of active agent (e.g. rGSH) administered to a subject over a 24 hour period of time. The daily dose can be administered two or more administrations during the 24 hour period. In one embodiment, the daily dose provides for two administrations in a 24 hour period. With this in mind, an “initial dose” or initial daily dose” refers to a dose administered during the initial regimen or period of a dosing regimen.

As used herein, “non-liquid” when used to refer to the state of a composition disclosed herein refers to the physical state of the composition as being a semi-solid or solid.

As used herein, “solid” and “semi-solid” refers to the physical state of a composition that supports its own weight at standard temperature and pressure and has adequate viscosity or structure to not freely flow. Semi-solid materials may conform to the shape of a container under applied pressure.

As used herein, “titration” or “dose titration” or “dose adjustment” are used interchangeably and refer to an increase or decrease of the total daily dose of rGSH administered to a subject, typically based on the response of the subject to the exogenous administered rGSH. The dose can be increased or decreased based on the measurement of serum rGSH concentration after a steady state has been achieved.

As used herein, “steady state” refers to the achievement of stable serum total rGSH levels upon a continuous dosing regimen (e.g. once daily, twice daily etc.) of the administered rGSH at a given dose, after at least 7 consecutive days (typically achieved after at least 15 days), following the start of the dosing regimen. Unless otherwise stated, steady states values set forth herein refer to steady states achieved after a final dose titration (i.e., no additional titrations are required), including situations where no dose titration is required. Similarly, as used herein, the “steady state serum concentration (Css, Css)” or “mean steady state serum concentration (mean Css)” of rGSH refers to the achievement of a stable serum total rGSH concentration in a subject or group of subjects, respectively, in response to a continuous dosing regimen (e.g. once daily, twice daily etc.) of the administered rGSH at a given dose, after at least 7 days (typically achieved after at least 15 days), following the start of the dosing regimen. It should be further noted that when a dose adjustment (increase or decrease in total daily dose of rGSH administered) is made as part of the dose-titration during the treatment, the mean Css is achieved at least about 7 days after the initiation of the change in the dose administered.

As used herein, the terms “release” and “release rate” are used interchangeably to refer to the discharge or liberation of a substance, including without limitation a drug, from the dosage form into a surrounding environment such as an aqueous medium either in vitro or in vivo.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, the term “delayed release” refers to the release into an aqueous solution of the rGSH from the composition or oral dosage form in a time delayed manner attributed either to the inherent nature of the composition or to a coating which may surround the composition or the oral dosage form. In one embodiment, the delayed release is such that about 20% or less of the rGSH is released within the first 15 minutes after the composition is contacted by the aqueous solution.

The terms “serum rGSH levels,” “plasma rGSH concentration,” “rGSH concentration in the blood,” and “serum rGSH concentration,” are used interchangeably and refer to the “total” rGSH concentration which is the sum of the bioavailable rGSH including free and bound (if any) rGSH concentrations. Unless otherwise specified, these values are “observed rGSH concentrations without adjusting or correcting for the baseline serum rGSH levels in the subject(s). As with any bio-analytical measure, for increased consistency the method employed to measure initial serum rGSH levels should be consistent with the method used to monitor and re-measure serum rGSH levels during clinical testing and rGSH therapy for a subject. Unless otherwise stated, “rGSH concentration” refers to serum total rGSH concentration.

As used herein, the average serum rGSH concentration can be determined using methods and practices known in the art. For example, the average baseline plasma rGSH concentration of a subject can be the arithmetic mean of the total plasma rGSH concentrations determined on at least two consecutive time points that are reasonably spaced from each other, for example from about 1 hour to about 168 hours apart. In a particular case, the plasma rGSH concentration can be determined on at least two consecutive times that are about 12 hours to about 48 hours apart. In another particular method, the plasma rGSH concentration of the subject can be determined at a time between about 5 o'clock and about 11 o'clock in the morning. Further, the plasma rGSH concentration can be determined by standard analytical procedures and methods available in the art, such as for example, automated or manual immunoassay methods, liquid chromatography or liquid chromatography-tandem mass spectrometry (LC-MSMS) etc.

As used herein, the term AUCₜ₁₋ₜ₂ is the area under the curve of a plasma-versus-time graph determined for the analyte from the time “t1 to time t2”. Wherein t1 and t2 are times (in hours) post dosing. For Example, t1 could be 1 hour and t2 could be 2 hours.

As used herein, the term “C_{avg},” “Cₐᵥₑ,” or “C-average” are used interchangeably, and is determined as the AUCₜ₁₋ₜ₂mean AUC divided by the time period (|t1−t2|). For example, C_{avg t0-t8} is the average plasma concentration over a period of 8 hours from t1=0 to t2=8 hours) post-dosing determined by dividing the AUC t₀₋ₜ₈ value by 8. Similarly, C_{avg t0-t12} is the average plasma concentration over a period of 12 hours post-dosing determined by dividing the AUCt₀₋ₜ₁₂ value by 12 (t1=042=12). Similarly, C_{avg t12-t24} is the average plasma concentration over a period of 12 hours post-dosing determined by dividing the AUCₜ₁₂₋ₜ₂₄ value by 12 (t1=1242=24); C_{avg-t24} is the average plasma concentration over a period of 24 hours post-dosing determined by dividing the AUCt₀₋ₜ₂₄ value by 24 (t1=0−t2=24), and so on. Unless otherwise stated, all C_{avg} values are considered to be C_{avg-t24} and unless otherwise stated, all the time values are expressed in hours (h). For example, the term C_{avg t0-t24} denotes C_{avg} from time zero (0) to 24 hours post dosing.

As used herein, “Cₜ” refers to the serum concentration of rGSH at time “t” prior to or after administration of the dosage of the current disclosure. The time “t” is generally in hours, unless otherwise specified. For example, a Cₜ of “C_{(-2 to 0)} refers to serum rGSH concentration measured in sample collected between the time of about 2 hours before and just immediately prior to dosage administration to the subject tested. Similarly, Cₜ of “C_{(2 to 4)}” refers to serum rGSH concentration measured in sample collected between the time of about 2 hours and 4 hours after administration of a dosage to the subject tested.

As used herein “single unit” when used to describe dosing of a subject refers to the dosage form being a single dosage form, e.g. a single tablet, capsule, pump or squirt of gel or solution, etc. In contrast, “multiple unit” when used to describe dosing of a subject refers to the dosage including two or more dosage forms, e.g. 2 tablets, 3 capsules, 2-4 pumps or squirts, etc. It is noteworthy that multiple unit dosage forms generally will be the same type of dosage forms (i.e. tablet or capsule) but are not required to be the same dosage form type.

In this disclosure, “comprises,” “comprising,” “containing” and “having” and the like can have the meaning ascribed to them in U.S. patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. patent law. “Consisting essentially of” or “consists essentially of” have the meaning generally ascribed to them by U.S. patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” “maximized,” “minimized,” and the like refer to a property of a device, component, composition, or activity that is measurably different from other devices, components, compositions or activities that are in a surrounding or adjacent area, that are similarly situated, that are in a single device or composition or in multiple comparable devices or compositions, that are in a group or class, that are in multiple groups or classes, or as compared to the known state of the art. For example, a composition that has “increased” stability of rGSH keeps a higher amount of its glutathione content in a reduced form (rGSH) rather than converting to an oxidized form (GSSG) for the same or greater amount of time as compared to a similar composition which does not achieve such results.

The term “coupled,” as used herein, is defined as directly or indirectly connected in a chemical, mechanical, electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Reference will now be made in detail to preferred embodiments of the disclosure. While the disclosure will be described in conjunction with the preferred embodiments, it will be understood that it is not intended to limit the disclosure to those preferred embodiments. To the contrary, it is intended to cover alternatives, variants, modifications, and equivalents as may be included within the spirit and scope of the disclosure as defined by the appended claims.

With the above background in mind, the inventors have identified a need for an efficient and patient-friendly mechanism or way to help increase and/or restore rGSH levels in a subject. For example, compositions, dosage forms and methods of treatment have been discovered which are capable of increasing serum rGSH levels in most patients for longer periods of time post dosing in a larger percentage of patients. Further, the compositions, dosage forms and methods of treatment disclosed herein are able to accomplish these desirable results while still providing patient-friendly regimens—such as, a practical rGSH equivalent daily dose, topical and transmucosal dosage forms, and fewer number of dosage units per administration.

It has been discovered that reduced glutathione (rGSH) and a substantially α-D-glucopyranoside-free stabilizing carrier, such as disclosed in Applicant's co-pending U.S. Provisional Patent Application filed on Apr. 30, 2020 under Attorney Docket No. 4515-001.PROV, which is incorporated herein by reference, is suitable to provide sustained, safe and effective increased rGSH levels relative to baseline levels through effective dosing regimens (dose, dosing frequency, dosage units). Accordingly, it has been found that a dose of rGSH and a substantially α-D-glucopyranoside-free stabilizing carrier can offer adequate bioactivity and bioavailability as compared to rGSH, and maintain sustained rGSH levels in a subject. Moreover, the compositions/dosage forms of rGSH and a substantially α-D-glucopyranoside-free stabilizing carrier at their daily dose when administered to a group of subjects, result in increased baseline levels of rGSH for the majority of subjects.

It has been further discovered that a combination of deoxygenated water and rGSH for example, as disclosed in Applicant's issued U.S. Pat. No. 10,272,130, which is incorporated herein by reference, is suitable to provide sustained, safe and effective increased rGSH levels relative to baseline levels through effective dosing regimens (dose, dosing frequency, dosage units). Accordingly, it has been found that a dose of a combination of deoxygenated water and rGSH can offer increased bioactivity and bioavailability as compared to baseline levels, and maintain increased rGSH levels in a subject. Moreover, the compositions/dosage forms of a combination of deoxygenated water and rGSH at their daily dose when administered to a group of subjects, results in increased baseline levels of rGSH for the majority of subjects.

Methods:

Methods of treating conditions responsive to treatment with reduced glutathione (rGSH) therapy are disclosed and described. Generally speaking, such methods can include administering a therapeutically effective amount of rGSH to a subject using a stabilized composition or dosage form (e.g. a composition or dosage form that stabilizes glutathione and prevents or reduces oxidation from a reduced state to an oxidized state, for example from rGSH to GSSG).

In one embodiment, a method of treating a subject having a condition responsive to treatment with reduced glutathione (rGSH) can comprise administering a therapeutically effective amount of a substantially α-D-glucopyranoside-free stabilized rGSH composition to the subject. In one example, more than about 80% of the rGSH can remain in a reduced form upon administration to the subject. In another example, more than about 50% of the rGSH can remain in a reduced form upon reaching a situs of action in the subject.

Skin Conditions

In one example, the condition can be a skin condition. The skin condition can be selected from the group consisting of: psoriasis, eczema, acne, hives, warts, cold sores, candidiasis, athlete's foot, wounds, mouth wounds, surgical mouth wounds, mouth wounds after oral surgery, mouth pain, burns, sunburns, dry skin, wrinkles, blisters, actinic keratosis, rosacea, carbuncles, cellulitis, contact dermatitis, and keratosis pilaris.

In one embodiment, the rGSH can be administered in an amount of from about 50 mg to about 1500 mg. In another example, the rGSH can be administered in an amount of from about 200 mg to about 1200 mg. In another example, the rGSH can be administered in an amount of from about 300 mg to about 800 mg. In another example, the rGSH can be administered in an amount of from about 400 mg to about 600 mg. In another example, the rGSH can be administered in an amount of from about 300 mg to about 500 mg. In another example, the rGSH can be administered in an amount of from about 600 mg to about 800 mg. While these ranged can apply to the various skin conditions recited above, in one embodiment, the skin condition can be a burn. In another embodiment, the skin condition can be a wound. In yet another embodiment, the skin condition is eczema.

The method of treating skin conditions can further comprise administering the composition to the subject according to a dosage regimen. In one embodiment, the dosage regimen can comprise administering the composition to the subject at least once per day for a specified duration. The dosage regimen can also comprise administering the composition to the subject multiple times per day for a specified duration. The duration can be from a single day to a month.

In some embodiments, administration of the rGSH can reduce or alleviate the symptoms of the skin condition by at least about 10% as compared to prior to commencement of the treatment. In another example, the symptoms of the condition can be reduced by at least 20% since commencement of the treatment. In another example, the symptoms of the condition can be reduced by at least 40% since commencement of the treatment. In another example, the symptoms of the condition can be reduced by at least 60% since commencement of the treatment. In another example, the symptoms of the condition can be reduced by at least 80% since commencement of the treatment.

In one embodiment, the composition for treating a skin condition can be in a topical dosage form. In some aspects, the composition can further comprise a stabilizing carrier. In one example, the stabilizing carrier can comprise or consist essentially of deoxygenated water. In another example, the stabilizing carrier can further comprise a solution, a suspension, an emulsion, a gel, a hydrogel, a thermo-responsive gel, a cream, a paste, or an ointment.

In one embodiment of a dosage form as recited above that is suitable to treat a skin condition, the therapeutically effective amount of rGSH can be from about 50 mg to about 1500 mg. In another example, the rGSH can be present in an amount of from about 200 mg to about 1200 mg. In another example, the rGSH can be present in an amount of from about 300 mg to about 800 mg. In yet another example, the rGSH can be present in an amount of from about 400 mg to about 600 mg. In another example, the rGSH can be administered in an amount of from about 300 mg to about 500 mg. In another example, the rGSH can be administered in an amount of from about 600 mg to about 800 mg.

In another embodiment, a method of treating a skin condition can further comprise co-administering an additional active agent to the subject. In some aspects, such co-administration can be concomitant administration. In some aspects, the active agent can be in the same or a different composition as the rGSH. In some aspects, the additional active agent can be a member selected from the group consisting of: an antioxidant, an antibiotic, an antiviral, an antitoxin agent, hydroxytyrosol (HXT), superoxide dismutase (SOD), catalase, an anti-infective agent, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof. When the condition is a burn or a wound, the active agent can be an antibiotic. When the condition is eczema, the active agent can be an antioxidant.

Neurologic Conditions

In one example, the condition to be treated can be a neurological condition. In some aspects, the neurological condition can be symptomized by a motor effect. When the neurological condition is symptomized by a motor effect, the neurological condition can be selected from the group consisting of: tardive dyskinesia, amyotrophic lateral sclerosis (ALS), primary lateral sclerosis (PLS), progressive muscular atrophy (PMA), progressive bulbar palsy, pseudobulbar palsy, monomelic amyotrophy (MMA), Bell's palsy, cerebral palsy, multiple sclerosis (MS), muscular dystrophy, parkinson's disease, and vertigo.

In some embodiments, the neurological condition can be symptomized by pain. When the neurological condition is symptomized by pain, the neurological condition can be selected from the group consisting of: shingles, peripheral neuropathy, chemotherapy-induced peripheral neuropathy, diabetic peripheral neuropathy, concussion, neuralgia, copper deficiency myeloneuropathy, diabetic amyotrophy, hypothyroidism, paraneoplastic sensory neuronopathy, uremic neuropathy, carpal tunnel syndrome, herpes zoster, and median neuropathy.

In some embodiments, the neurological condition can be symptomized by a neurocognitive effect. When the neurological condition is symptomized by a neurocognitive effect, the neurological condition can be selected from the group consisting of: familial Alzheimer's disease, SCA17 (dominant inheritance); adrenoleukodystrophy (X-linked); Gaucher's disease type 3, metachromatic leukodystrophy, Niemann-Pick disease type C, pantothenate kinase-associated neurodegeneration, Tay-Sachs disease, Wilson's disease, Alzheimer's disease, vascular dementia, dementia with Lewy bodies, frontotemporal dementia, Creutzfeldt-Jakob disease, Behcet's disease, multiple sclerosis, sarcoidosis, Sjogren's syndrome, systemic lupus erythematosus, celiac disease, non-celiac gluten sensitivity, Alexander disease, canavan disease, cerebrotendinous xanthomatosis, dentatorubral-pallidoluysian atrophy, epilepsy, fatal familial insomnia, fragile X-associated tremor/ataxia syndrome, glutaric aciduria type 1, Krabbe's disease, Maple syrup urine disease, Niemann-Pick disease type C, Neuronal ceroid lipofuscinosis, Neuroacanthocytosis, Organic acidemias, Pelizaeus-Merzbacher disease, Sanfilippo syndrome type B, Spinocerebellar ataxia type 2, urea cycle disorders, alcohol dementia, Wernicke's encephalopathy, Korsakoffs psychosis, and chronic traumatic encephalopathy, and combinations thereof.

In some embodiments, the neurocognitive effect can be associated with decreased production of rGSH or decreased recycling of rGSH. In one example, the decreased production or recycling of rGSH can be associated with genetic errors in a gene selected from: APOE4, APP, PSEN1, PSEN2, PLD3, GSTO1, GSTM3, GSTP1, and combinations thereof.

In one embodiment, the rGSH can be administered in an amount of from about 50 mg to about 1500 mg. In another example, the rGSH can be administered in an amount of from about 200 mg to about 1200 mg. In another example, the rGSH can be administered in an amount of from about 300 mg to about 800 mg. In another example, the rGSH can be administered in an amount of from about 400 mg to about 600 mg. In another example, the rGSH can be administered in an amount of from about 300 mg to about 500 mg. In another example, the rGSH can be administered in an amount of from about 600 mg to about 800 mg. While these ranged can apply to the various neurologic conditions recited above, in one embodiment, the neurologic condition can be a shingles. In another embodiment, the neurologic condition can be tardive dyskinesia. In yet another embodiment, the condition is neuropathy, such as chemotherapy-induced or diabetic neuropathy.

The method of treating a neurologic condition can further comprise administering the composition to the subject according to a dosage regimen. In one aspect, the dosage regimen can comprise administering the composition to the subject at least once per day for a specified duration. In another aspect, the dosage regimen can comprise administering the composition to the subject multiple times per day for a specified duration. Furthermore, the duration can be from a single day to a month.

In some embodiments, the symptoms of the neurologic condition can be reduced by at least about 10% as compared to prior to commencement of the treatment. In another example, the symptoms of the condition can be reduced by at least 20% since commencement of the treatment. In another example, the symptoms of the condition can be reduced by at least 40% since commencement of the treatment. In another example, the symptoms of the condition can be reduced by at least 60% since commencement of the treatment. In another example, the symptoms of the condition can be reduced by at least 80% since commencement of the treatment.

In an example, the method of treating a subject with a neurologic condition can include administering a topical dosage form or an oral dosage form or a combination thereof. The composition can further comprise a stabilizing carrier. In one example, the stabilizing carrier can comprise deoxygenated water. In one example, the stabilizing carrier can consist essentially of deoxygenated water.

When the composition is a topical dosage form, the stabilizing carrier can further comprise a solution, a suspension, an emulsion, a gel, a hydrogel, a thermo-responsive gel, a cream, a paste, or an ointment.

In one embodiment of a dosage form as recited above that is suitable to treat a neurologic condition, the therapeutically effective amount of rGSH can be from about 50 mg to about 1500 mg. In another example, the rGSH can be present in the composition in an amount of from about 200 mg to about 1200 mg. In another example, the rGSH can be present in the composition in an amount of from about 300 mg to about 800 mg. In another example, the rGSH can be present in the composition in an amount of from about 400 mg to about 600 mg. In another example, the rGSH can be administered in an amount of from about 300 mg to about 500 mg. In another example, the rGSH can be administered in an amount of from about 600 mg to about 800 mg.

When the composition is an oral dosage form, the stabilizing carrier can further comprise binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof.

In an example, the method of treating a neurological condition can further comprise co-administering an active agent to the subject. The co-administration can be concomitant administration. The active agent can be in the same or a different composition as the rGSH. The active agent can be a member selected from the group consisting of: an antioxidant, an antibiotic, an antiviral, an antitoxin agent, hydroxytyrosol (HXT), superoxide dismutase (SOD), catalase, an anti-infective agent, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof. When the condition is tardive dyskinesia, the additional active agent can be an antioxidant. When the condition is shingles, the additional active agent can be an analgesic.

The dosage regimen can comprise administering the composition to the subject at least once per day for a specified duration. The dosage regimen can comprise administering the composition to the subject multiple times per day for a specified duration. The duration can be from a single day to a month.

Inflammatory Conditions

In one example, the condition to be treated can be an inflammatory condition. In some aspects, the inflammatory condition can be selected from the group consisting of: fibromyalgia, diabetes, arthritis, chronic obstructive pulmonary disease, rheumatoid arthritis, bronchitis, appendicitis, asthma, ulcers, tuberculosis, periodontitis, ulcerative colitis, Crohn's disease, sinusitis, hepatitis, hepatitis A, hepatitis B, hepatitis C, hepatitis D, hepatitis E, temporal arteritis, inflammatory bowel disease, and allergies.

In some embodiments, the rGSH can be administered in an amount of from about mg to about 1500 mg. In another example, the rGSH can be administered in an amount of from about 200 mg to about 1200 mg. In another example, the rGSH can be administered in an amount of from about 300 mg to about 800 mg. In another example, the rGSH can be administered in an amount of from about 400 mg to about 600 mg. In another example, the rGSH can be administered in an amount of from about 300 mg to about 500 mg. In another example, the rGSH can be administered in an amount of from about 600 mg to about 800 mg. While these ranges can apply to the various inflammatory conditions recited above, in one embodiment, the inflammatory condition is fibromyalgia.

The method of treating an inflammatory condition can further comprise administering the composition to the subject according to a dosage regimen. In one aspect, the dosage regimen can comprise administering the composition to the subject at least once per day for a specified duration. In another aspect, the dosage regimen can comprise administering the composition to the subject multiple times per day for a specified duration. Furthermore, the duration can be from a single day to a month.

In some embodiments, the symptoms of the inflammatory condition can be reduced by at least about 10% as compared to prior to commencement of the treatment. In another example, the symptoms of the condition can be reduced by at least 20% as compared to prior to commencement of the treatment. In another example, the symptoms of the condition can be reduced by at least 40% as compared to prior to commencement of the treatment. In another example, the symptoms of the condition can be reduced by at least 60% as compared to prior to commencement of the treatment. In another example, the symptoms of the condition can be reduced by at least 80% as compared to prior to commencement of the treatment.

In an example, the method of treating a subject with an inflammatory condition can include administering a topical dosage form or an oral dosage form or a combination thereof. The composition can further comprise a stabilizing carrier. In one example, the stabilizing carrier can comprise deoxygenated water. In one example, the stabilizing carrier can consist essentially of deoxygenated water.

When the composition is an oral dosage form, the stabilizing carrier can further comprise binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof.

In one embodiment of a dosage form as recited above that is suitable to treat a neurologic condition, the therapeutically effective amount of rGSH can be from about 50 mg to about 1500 mg. In another example, the rGSH can be present in the composition in an amount of from about 200 mg to about 1200 mg. In another example, the rGSH can be present in the composition in an amount of from about 300 mg to about 800 mg. In another example, the rGSH can be present in the composition in an amount of from about 400 mg to about 600 mg. In another example, the rGSH can be administered in an amount of from about 300 mg to about 500 mg. In another example, the rGSH can be administered in an amount of from about 600 mg to about 800 mg.

When the composition is a topical dosage form, the stabilizing carrier can further comprise a solution, a suspension, an emulsion, a gel, a hydrogel, a thermo-responsive gel, a cream, a paste, or an ointment.

In an example, the method of treating an inflammatory condition can further comprise co-administering an active agent to the subject. The co-administration can be concomitant administration. The active agent can be in the same or a different composition as the rGSH. The active agent can be a member selected from the group consisting of: an antioxidant, an antibiotic, an antiviral, an antitoxin agent, hydroxytyrosol (HXT), superoxide dismutase (SOD), catalase, an anti-infective agent, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

When the condition is fibromyalgia, the active agent can be an antioxidant, and/or a member selected from the group consisting of: molybdenum, boron, zinc chelate, N-Acetyl L-Cysteine, L-serine, and L-glutamine, pyridoxine, folate, calcium pantothenate, trimethyl glycine, L-serine, N-acetyl L-tyrosine, methyl-cobalamin, calcium folinate, pantothenic acid, choline-L-bitartrate, and combinations thereof.

Virally-Induced Conditions

In one example, the condition to be treated can be a virally-induced condition. In some aspects, the virally-induced condition can be selected from the group consisting of: shingles, post-herpetic neuralgia (PHN), HIV, herpes simplex encephalitis, chickenpox, measles, rubella, roseola, meningitis, encephalitis, warts, oral herpes, genital herpes, hepatitis, norovirus, rotavirus, adenovirus, flu, astrovirus, common cold, respiratory syncytial virus, parainfluenza, severe acute respiratory syndrome (SARS), middle east respiratory syndrome (MERS), coronavirus, and combinations thereof.

In one embodiment, the rGSH can be administered in an amount of from about 50 mg to about 1500 mg. In another example, the rGSH can be administered in an amount of from about 200 mg to about 1200 mg. In another example, the rGSH can be administered in an amount of from about 300 mg to about 800 mg. In another example, the rGSH can be administered in an amount of from about 400 mg to about 600 mg. In another example, the rGSH can be administered in an amount of from about 300 mg to about 500 mg. In another example, the rGSH can be administered in an amount of from about 600 mg to about 800 mg. While these ranges can apply to the various virally-induced conditions recited above, in one embodiment, the virally-induced condition can be shingles. In another embodiment, the virally-induced condition can be post-herpetic neuralgia.

The method of treating a virally-induced condition can further comprise administering the composition to the subject according to a dosage regimen. In one aspect, the dosage regimen can comprise administering the composition to the subject at least once per day for a specified duration. In another aspect, the dosage regimen can comprise administering the composition to the subject multiple times per day for a specified duration. Furthermore, the duration can be from a single day to a month.

In some embodiments, the symptoms of the virally-induced condition can be reduced by at least about 10% as compared to prior to commencement of the treatment. In another example, the symptoms of the condition can be reduced by at least 20% as compared to prior to commencement of the treatment. In another example, the symptoms of the condition can be reduced by at least 40% as compared to prior to commencement of the treatment. In another example, the symptoms of the condition can be reduced by at least 60% as compared to prior to commencement of the treatment. In another example, the symptoms of the condition can be reduced by at least 80% as compared to prior to commencement of the treatment.

In an example, the method of treating a subject with a virally-induced condition can include administering a topical dosage form, an oral dosage form, a transmucosal dosage form, or a combination thereof. The composition can further comprise a stabilizing carrier. In one example, the stabilizing carrier can comprise deoxygenated water. In one example, the stabilizing carrier can consist essentially of deoxygenated water.

When the composition is an oral dosage form, the stabilizing carrier can further comprise binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof.

In an embodiment of a dosage form as recited above that is suitable to treat a virally-induced condition, the therapeutically effective amount of rGSH can be from about 50 mg to about 1500 mg. In another example, the rGSH can be present in the composition in an amount of from about 200 mg to about 1200 mg. In another example, the rGSH can be present in the composition in an amount of from about 300 mg to about 800 mg. In another example, the rGSH can be present in the composition in an amount of from about 400 mg to about 600 mg. In another example, the rGSH can be administered in an amount of from about 300 mg to about 500 mg. In another example, the rGSH can be administered in an amount of from about 600 mg to about 800 mg.

When the composition is a topical dosage form, the stabilizing carrier can further comprise a solution, a suspension, an emulsion, a gel, a hydrogel, a thermo-responsive gel, a cream, a paste, or an ointment.

When the composition is a transmucosal dosage form, the stabilizing carrier can further comprise a solution, an adhesive layer, or a bio-erodible matrix.

In an example, the method of treating a virally-induced condition can further comprise co-administering an active agent to the subject. The co-administration can be concomitant administration. The active agent can be in the same or a different composition as the rGSH. The active agent can be a member selected from the group consisting of: an antioxidant, an antibiotic, an antiviral, an antitoxin agent, hydroxytyrosol (HXT), superoxide dismutase (SOD), catalase, an anti-infective agent, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof. When the condition is shingles, the active agent can be an analgesic. When the condition is post-herpatic neuropathy, the active agent can be an anti-viral.

Antioxidant Conditions

In one example, the condition to be treated can be responsive to treatment with an antioxidant. In one embodiment, the rGSH can be administered in an amount of from about 50 mg to about 1500 mg. In another example, the rGSH can be administered in an amount of from about 200 mg to about 1200 mg. In another example, the rGSH can be administered in an amount of from about 300 mg to about 800 mg. In another example, the rGSH can be administered in an amount of from about 400 mg to about 600 mg. In another example, the rGSH can be administered in an amount of from about 300 mg to about 500 mg. In another example, the rGSH can be administered in an amount of from about 600 mg to about 800 mg.

The method of treating a condition responsive to treatment with an antioxidant can further comprise administering the composition to the subject according to a dosage regimen. In one aspect, the dosage regimen can comprise administering the composition to the subject at least once per day for a specified duration. In another aspect, the dosage regimen can comprise administering the composition to the subject multiple times per day for a specified duration. Furthermore, the duration can be from a single day to a month.

In some embodiments, the symptoms of the condition can be reduced by at least about 10% as compared to prior commencement of the treatment. In another example, the symptoms of the condition can be reduced by at least 20% as compared to prior commencement of the treatment. In another example, the symptoms of the condition can be reduced by at least 40% as compared to prior commencement of the treatment. In another example, the symptoms of the condition can be reduced by at least 60% as compared to prior commencement of the treatment. In another example, the symptoms of the condition can be reduced by at least 80% as compared to prior commencement of the treatment.

In an example, the method of treating a subject with a condition responsive to an antioxidant can include administering an oral dosage form. The composition can further comprise a stabilizing carrier. In one example, the stabilizing carrier can comprise deoxygenated water. In one example, the stabilizing carrier can consist essentially of deoxygenated water.

When the composition is an oral dosage form, the stabilizing carrier can further comprise binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof.

In one embodiment of a dosage form as recited above that is suitable to treat a condition responsive to an antioxidant, the therapeutically effective amount of rGSH can be from about 50 mg to about 1500 mg. In another example, the rGSH can be present in the composition in an amount of from about 200 mg to about 1200 mg. In another example, the rGSH can be present in the composition in an amount of from about 300 mg to about 800 mg. In another example, the rGSH can be present in the composition in an amount of from about 400 mg to about 600 mg. In another example, the rGSH can be administered in an amount of from about 300 mg to about 500 mg. In another example, the rGSH can be administered in an amount of from about 600 mg to about 800 mg.

In an example, the method of treating a condition responsive to an antioxidant can further comprise co-administering an active agent to the subject. The co-administration can be concomitant administration. The active agent can be in the same or a different composition as the rGSH. The active agent can be a member selected from the group consisting of: an antioxidant, an antibiotic, an antiviral, an antitoxin agent, hydroxytyrosol (HXT), superoxide dismutase (SOD), catalase, an anti-infective agent, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

Immune-Stimulating Response

In one example, the method can further comprise stimulating an immune response. In some aspects, the rGSH can be administered in an amount of from about 50 mg to about 1500 mg. In another example, the rGSH can be administered in an amount of from about 200 mg to about 1200 mg. In another example, the rGSH can be administered in an amount of from about 300 mg to about 800 mg. In another example, the rGSH can be administered in an amount of from about 400 mg to about 600 mg. In another example, the rGSH can be administered in an amount of from about 300 mg to about 500 mg. In another example, the rGSH can be administered in an amount of from about 600 mg to about 800 mg.

The method of treating stimulating an immune response can further comprise administering the composition to the subject according to a dosage regimen. In one aspect, the dosage regimen can comprise administering the composition to the subject at least once per day for a specified duration. In another aspect, the dosage regimen can comprise administering the composition to the subject multiple times per day for a specified duration. Furthermore, the duration can be from a single day to a month.

In some embodiments, the stimulated immune response can reduce the symptoms of the condition by at least about 10% since commencement of the treatment. In another example, the symptoms of the condition can be reduced by at least 20% since commencement of the treatment. In another example, the symptoms of the condition can be reduced by at least 40% since commencement of the treatment. In another example, the symptoms of the condition can be reduced by at least 60% since commencement of the treatment. In another example, the symptoms of the condition can be reduced by at least 80% since commencement of the treatment.

In an example, the method of treating a stimulating an immune response in a subject can include administering an oral dosage form. The composition can further comprise a stabilizing carrier. In one example, the stabilizing carrier can comprise deoxygenated water. In one example, the stabilizing carrier can consist essentially of deoxygenated water.

When the composition is an oral dosage form, the stabilizing carrier can further comprise binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof.

In an example, the method of stimulating an immune response in a subject can further comprise co-administering an active agent to the subject. The co-administration can be concomitant administration. The active agent can be in the same or a different composition as the rGSH. The active agent can be a member selected from the group consisting of: an antioxidant, an antibiotic, an antiviral, an antitoxin agent, hydroxytyrosol (HXT), superoxide dismutase (SOD), catalase, an anti-infective agent, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof

Toxins:

In one example, the condition to be treated can be induced by toxins. In some aspects, the condition can be selected from the group consisting of: acetaminophen overdose, narcotic overdose, peanut allergies, alcohol toxicity, paracetamol toxicity, heavy-metal poisoning, insect bites, poison ivy reactions, anaphylaxis, and combinations thereof.

In one embodiment, the rGSH can be administered in an amount of from about 50 mg to about 1500 mg. In another example, the rGSH can be administered in an amount of from about 200 mg to about 1200 mg. In another example, the rGSH can be administered in an amount of from about 300 mg to about 800 mg. In another example, the rGSH can be administered in an amount of from about 400 mg to about 600 mg. In another example, the rGSH can be administered in an amount of from about 300 mg to about 500 mg. In another example, the rGSH can be administered in an amount of from about 600 mg to about 800 mg.

The method of treating a toxin-induced condition can further comprise administering the composition to the subject according to a dosage regimen. In one aspect, the dosage regimen can comprise administering the composition to the subject at least once per day for a specified duration. In one aspect, the dosage regimen can comprise administering the composition to the subject multiple times per day for a specified duration. Furthermore, the duration can be from a single day to a month.

In some embodiments, the symptoms of the toxin-induced condition can be reduced by at least about 10% since commencement of the treatment. In another example, the symptoms of the condition can be reduced by at least 20% since commencement of the treatment. In another example, the symptoms of the condition can be reduced by at least 40% since commencement of the treatment. In another example, the symptoms of the condition can be reduced by at least 60% since commencement of the treatment. In another example, the symptoms of the condition can be reduced by at least 80% since commencement of the treatment.

In an example, the method of treating a subject with a toxin-induced condition can include administering a parenteral dosage form. The composition can further comprise a stabilizing carrier. In one example, the stabilizing carrier can comprise deoxygenated water. In one example, the stabilizing carrier can consist essentially of deoxygenated water.

The parenteral dosage form can further comprise one or more of water, a tonicity agent, a buffering agent, a preservative, or a combination thereof.

In one embodiment of a dosage form as recited above that is suitable to treat a toxin-induced condition, the therapeutically effective amount of rGSH can be from about 1 mg to about 1500 mg. In another example, the rGSH can be present in the composition in an amount of from about 5 mg to about 250 mg. In another example, the rGSH can be present in the composition in an amount of from about 10 mg to about 100 mg. In another example, the rGSH can be present in the composition in an amount of from about 25 mg to about 50 mg.

In an example, the method of treating a toxin-induced condition can further comprise co-administering an active agent to the subject. The co-administration can be concomitant administration. The active agent can be in the same or a different composition as the rGSH. The active agent can be a member selected from the group consisting of: an antioxidant, an antibiotic, an antiviral, an antitoxin agent, hydroxytyrosol (HXT), superoxide dismutase (SOD), catalase, an anti-infective agent, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

In another example, more than about 80% of the rGSH can remain in a reduced form upon administration to the subject. In another example, more than about 50% of the rGSH can remain in a reduced form upon administration to the subject. more than about 65% of the rGSH can remain in a reduced form upon administration to the subject. more than about 90% of the rGSH can remain in a reduced form upon administration to the subject. more than about 95% of the rGSH can remain in a reduced form upon administration to the subject.

In another example, more than about 50% of the rGSH can remain in a reduced form upon reaching a situs of action in the subject. In another example, more than about 20% of the rGSH can remain in a reduced form upon reaching a situs of action in the subject. In another example, more than about 70% of the rGSH can remain in a reduced form upon reaching a situs of action in the subject. In another example, more than about 90% of the rGSH can remain in a reduced form upon reaching a situs of action in the subject.

In one example, the condition can be selected from the group consisting of: acetaminophen overdose, drug-resistant bacterial infection, liver failure, HIV, herpetic encephalopathy, cystic fibrosis, narcotic overdose, macular degeneration, cataracts, glaucoma, peanut allergies, and combinations thereof.

In one example, the condition can be selected from the group consisting of: diabetic neuropathy, post-herpetic neuralgia (PHN), cold sore development, alcohol toxicity, paracetamol toxicity, wet macular degeneration, cataracts formation, corneal ulcers, teeth caries, teeth cavities, sunburns, laser burns, Epstein-Barr virus diseases, wounds, scarring, chronic renal failure (CRF), nephropathy, heavy-metal poisoning, chemotherapy-induced aphthous mouth ulcers, post tonsil surgery, insect bites, poison ivy reactions, anaphylaxis, necrobiosis lipoidica diabeticorum, pulmonary fibrosis, chronic obstructive pulmonary disease (COPD), optic neuritis (ON), Leber hereditary optic neuropathy (LHON)-like optic neuropathy (LLON), non-arteritic anterior ischemic optic neuropathy (NAION), sporadic bilateral optic neuropathy (SBON), and combinations thereof.

In another example, the condition can be G6PD (glucose-6-phosphate dehydrogenase) deficiency.

In one embodiment, the stabilizing agent can include an oxygen scavenger, a synthetic polymer, a natural non-α-D-glucopyranoside polymer, a lipid, a protein, an amino acid, a co-polymer, an inert gas, or a combination thereof.

In some examples, the stabilizing agent can include an oxygen scavenger. Non-limiting examples of oxygen scavengers can include, but are not limited to, suitable combinations of: a transition metal catalyst (e.g., copper), ascorbic acid, glucose oxidase, catalase, polyunsaturated fatty-acids, soybean oil, sesame oil, cottonseed oil, squalene, fatty acids, polybutadiene, yeast, antioxidants, the like, and combinations thereof. In some examples, the stabilizing agent can include a synthetic polymer. In some examples, the stabilizing agent can include a natural non-α-D-glucopyranoside polymer.

In another aspect, the stabilizing agent can be a lipid selected from the group consisting essentially of: phospholipids, glycolipids, cholesterol, triglycerides, fatty acids, fatty acid glycerides, surfactants, or a combination thereof. In an example, a phospholipid can include two hydrophobic fatty acid tails and a hydrophilic head consisting of a phosphate group. In an example, a phospholipid can be a glycerol-based phospholipid including, but not limited to, plasmalogens, phosphatidates, phosphatidylcholines, or glycolipids.

In another example, the lipid can form a liposome, an inverted micelle, or a combination thereof. Non-limiting examples of liposomes can include, but are not limited to, the multilamellar vesicle (MLV), the small unilamellar liposome vesicle (SUV), the large unilamellar vesicle (LUV), the cochleate vesicle, and combinations thereof.

In another example, the lipid can form an inverted micelle which can include nanometer-sized (1-10 nm) water droplets dispersed in a non-polar solvent obtained by the action of surfactants. Non-limiting examples of surfactants can include, but are not limited to, AOT [sodium bis(2-ethyl hexyl) sulfosuccinate], CTAB (cetyltrimethylammonium bromide), dode-cyl penta(oxyethylene) ether (C12E5), n-dodecyloctaoxyethylene glycol monoether (C12E8), cetylbenzyldimethylammonium chloride (CBAC), didodecyl-dimethylammonium bromide (DDAB), sorbitan monooleate, and sodium dodecylbenzene-sulfonate (NaDBS), Triton X-100[polyoxyethylene(10)isooctylphenyl ether], poly-oxyethylene(4) lauryl ether (known as Brij30), pentaoxyethylene-glycol-nonyl-phenyl ether (known as Igepal-00520), poly(oxyethylene)5nonylphenol ether (NP-5), poly(oxyethylene)9nonylphenol ether (NP-9), poly(oxyethylene)12 nonylphenol ether (NP-12), and combinations thereof.

In another embodiment, the compositions and the dosage forms can be formulated as one of a solution, a suspension, an emulsion, a gel, a hydrogel, a thermo-responsive gel, a cream, an ointment, a paste, an adhesive, an erodible matrix, a liquid reservoir, a patch, a powder, a compressed powder, or a combination thereof. The dosage forms of the current disclosure can comprise a transdermal dosage form, a parenteral dosage form, a topical dosage form, an oral dosage form, a nebulizer dosage form, a transmucosal dosage form, and combinations thereof.

### Examples

In one example, a method of treating a subject having a condition responsive to treatment with reduced glutathione (rGSH) can comprise: administering a therapeutically effective amount of a substantially α-D-glucopyranoside-free stabilized rGSH composition to the subject.

In one example, the condition can be a skin condition.

In one example, the skin condition can be selected from the group consisting of: psoriasis, eczema, acne, hives, warts, cold sores, candidiasis, athlete's foot, wounds, mouth wounds, mouth pain, burns, sunburns, dry skin, wrinkles, blisters, actinic keratosis, rosacea, carbuncles, cellulitis, contact dermatitis, and keratosis pilaris.

In one example, the skin condition can be a burn and the rGSH is present in the composition in an amount of from about 50 mg to about 1500 mg.

In one example, the condition can be a wound and the rGSH is present in the composition in an amount of from about 50 mg to about 1500 mg.

In one example, the condition can be eczema and the rGSH is present in the composition in an amount of from about 50 mg to about 1500 mg.

In one example, the method can comprise administering the composition to the subject according to a dosage regimen.

In one example, the dosage regimen can comprise administering the composition to the subject at least once per day for a specified duration.

In one example, the dosage regimen can comprise administering the composition to the subject multiple times per day for a specified duration.

In one example, the duration can be from a single day to a month.

In one example, the symptoms of the condition can be reduced by at least about 10% since commencement of the treatment.

In one example, the composition can be in a topical dosage form.

In one example, the composition can further comprise a stabilizing carrier.

In one example, the stabilizing carrier can comprise deoxygenated water.

In one example, the stabilizing carrier can consist essentially of deoxygenated water.

In one example, the stabilizing carrier can further comprise a solution, a suspension, an emulsion, a gel, a hydrogel, a thermo-responsive gel, a cream, a paste, or an ointment.

In one example, the carrier can comprise a solution.

In one example, the therapeutically effective amount of rGSH can be from about 50 mg to about 1500 mg.

In one example, the method can further comprise co-administering an active agent to the subject.

In one example, the co-administration can be concomitant administration.

In one example, the active agent can be in the same or a different composition as the rGSH.

In one example, the active agent can be a member selected from the group consisting of: an antioxidant, an antibiotic, an antiviral, an antitoxin agent, hydroxytyrosol (HXT), superoxide dismutase (SOD), catalase, an anti-infective agent, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

In one example, the condition can be a burn and the active agent is an antibiotic.

In one example, the condition can be a wound and the active agent is an antibiotic.

In one example, the condition can be eczema and the active agent is an antioxidant.

In one example, the condition can be a neurological condition.

In one example, the neurological condition can be symptomized by a motor effect.

In one example, the condition can be selected from the group consisting of: tardive dyskinesia, amyotrophic lateral sclerosis (ALS), primary lateral sclerosis (PLS), progressive muscular atrophy (PMA), progressive bulbar palsy, pseudobulbar palsy, monomelic amyotrophy (MMA), Bell's palsy, cerebral palsy, multiple sclerosis (MS), muscular dystrophy, parkinson's disease, and vertigo.

In one example, the condition can be tardive dyskinesia and the rGSH is present in the composition in an amount of from about 50 mg to about 1500 mg.

In one example, the neurological condition can be symptomized by pain.

In one example, the condition can be selected from the group consisting of: shingles, peripheral neuropathy, chemotherapy-induced peripheral neuropathy, diabetic peripheral neuropathy, concussion, neuralgia, copper deficiency myeloneuropathy, diabetic amyotrophy, hypothyroidism, paraneoplastic sensory neuronopathy, uremic neuropathy, carpal tunnel syndrome, herpes zoster, and median neuropathy.

In one example, the condition can be shingles and the rGSH is present in the composition in an amount of from about 50 mg to about 1500 mg.

In one example, the condition can be chemotherapy-induced peripheral neuropathy and the rGSH is present in the composition in an amount of from about 50 mg to about 1500 mg.

In one example, the condition can be diabetic peripheral neuropathy and the rGSH is present in the composition in an amount of from about 50 mg to about 1500 mg.

In one example, the method can further comprise administering the composition to the subject according to a dosage regimen.

In one example, the dosage regimen can comprise administering the composition to the subject at least once per day for a specified duration.

In one example, the dosage regimen can comprise administering the composition to the subject multiple times per day for a specified duration.

In one example, the duration can be from a single day to a month.

In one example, symptoms of the condition can be reduced by at least about 10% since commencement of the treatment.

In one example, the composition can be in a topical dosage form.

In one example, the composition can be in an oral dosage form.

In one example, the composition can further comprise a stabilizing carrier.

In one example, the stabilizing carrier can comprise deoxygenated water.

In one example, the stabilizing carrier can consist essentially of deoxygenated water.

In one example, the stabilizing carrier can further comprise one or more of: binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof.

In one example, the therapeutically effective amount of rGSH can be from about mg to about 1500 mg.

In one example, the method can further comprise co-administering an active agent to the subject.

In one example, the co-administration can be concomitant administration.

In one example, the active agent can be in the same or a different composition as the rGSH.

In one example, the active agent can be selected from the group consisting of: is a member selected from the group consisting of: an antioxidant, an antibiotic, an antiviral, an antitoxin agent, hydroxytyrosol (HXT), superoxide dismutase (SOD), catalase, an anti-infective agent, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

In one example, the condition can be tardive dyskinesia and the active agent is an antioxidant.

In one example, the condition can be shingles and the active agent is an anti-viral.

In one example, the condition can be chemotherapy-induced peripheral neuropathy and the active agent is an antioxidant.

In one example, the condition can be diabetic peripheral neuropathy and the active agent is an antioxidant.

In one example, the condition can be an inflammatory condition.

In one example, the inflammatory condition can be a member selected from the group consisting of: fibromyalgia, diabetes, arthritis, chronic obstructive pulmonary disease, rheumatoid arthritis, bronchitis, appendicitis, asthma, ulcers, tuberculosis, periodontitis, ulcerative colitis, Crohn's disease, sinusitis, hepatitis, temporal arteritis, inflammatory bowel disease, and allergies.

In one example, the condition can be fibromyalgia and the rGSH is present in the composition in an amount of from about 50 mg to about 1500 mg.

In one example, the method can further comprise administering the composition to the subject according to a dosage regimen.

In one example, the dosage regimen can comprise administering the composition to the subject at least once per day for a specified duration.

In one example, the dosage regimen can comprise administering the composition to the subject multiple times per day for a specified duration.

In one example, the duration can be from a single day to a month.

In one example, symptoms of the condition can be reduced by at least about 10% since commencement of the treatment.

In one example, the composition can be in an oral dosage form.

In one example, the composition can further comprise a stabilizing carrier.

In one example, the stabilizing carrier can comprise deoxygenated water.

In one example, the stabilizing carrier can consist essentially of deoxygenated water.

In one example, the stabilizing carrier can further comprise one or more of: binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof.

In one example, the carrier can comprise a taste-masking agent.

In one example, the therapeutically effective amount of rGSH can be from about 50 mg to about 1500 mg.

In one example, the method can further comprise co-administering an active agent to the subject.

In one example, the co-administration can be concomitant administration.

In one example, the active agent can be in the same or a different composition as the rGSH.

In one example, the active agent can be a member selected from the group consisting of: an antioxidant, an antibiotic, an antitoxin agent, an antiviral, hydroxytyrosol (HXT), superoxide dismutase (SOD), catalase, an anti-infective agent, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

In one example, the active agent can be a member selected from the group consisting of: molybdenum, boron, zinc chelate, N-Acetyl L-Cysteine, L-serine, and L-glutamine, pyridoxine, folate, calcium pantothenate, trimethyl glycine, L-serine, N-acetyl L-tyrosine, methyl-cobalamin, calcium folinate, pantothenic acid, choline-L-bitartrate, and combinations thereof.

In one example, the condition can be fibromyalgia and the active agent is an antioxidant.

In one example, the condition can be a virally-induced condition.

In one example, the viral condition can be a member selected from the group consisting of: shingles, post-herpetic neuralgia (PHN), HIV, herpes simplex encephalitis, chickenpox, measles, rubella, roseola, meningitis, encephalitis, warts, oral herpes, genital herpes, hepatitis, norovirus, rotavirus, adenovirus, flu, astrovirus, common cold, respiratory syncytial virus, parainfluenza, severe acute respiratory syndrome (SARS), middle east respiratory syndrome (MERS), coronavirus, and combinations thereof.

In one example, the condition can be shingles and the rGSH can be present in the composition in an amount of from about 50 mg to about 1500 mg.

In one example, the condition can be post-herpetic neuralgia and the rGSH can be present in the composition in an amount of from about 50 mg to about 1500 mg.

In one example, the method can further comprise administering the composition to the subject according to a dosage regimen.

In one example, the dosage regimen can comprise administering the composition to the subject at least once per day for a specified duration.

In one example, the dosage regimen can comprise administering the composition to the subject multiple times per day for a specified duration.

In one example, the duration can be from a single day to a month.

In one example, symptoms of the condition can be reduced by at least about 10% since commencement of the treatment.

In one example, the composition can be in a transmucosal dosage form.

In one example, the composition can further comprise a stabilizing carrier.

In one example, the stabilizing carrier can comprise deoxygenated water.

In one example, the stabilizing carrier can consist essentially of deoxygenated water.

In one example, the stabilizing carrier can further comprise a solution, an adhesive layer, or a bio-erodible matrix.

In one example, the carrier can comprise a solution.

In one example, the therapeutically effective amount of rGSH can be from about 50 mg to about 1500 mg.

In one example, the method can further comprise co-administering an active agent to the subject.

In one example, the co-administration can be concomitant administration.

In one example, the active agent can be in the same or a different composition as the rGSH.

In one example, the active agent can comprise a member selected from the group consisting of: an antiviral, an antioxidant, an antitoxin agent, N-acetyl-cysteine, hydroxytyrosol (HXT), superoxide dismutase (SOD), catalase, an anti-infective agent, an antibiotic, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

In one example, the condition can be shingles and the active agent is an antiviral.

In one example, the condition can be post-herpetic neuralgia and the active agent is an antiviral agent.

In one example, the condition can be responsive to treatment with an antioxidant.

In one example, the method can further comprise administering the composition to the subject according to a dosage regimen.

In one example, the dosage regimen can comprise administering the composition to the subject at least once per day for a specified duration.

In one example, the dosage regimen can comprise administering the composition to the subject multiple times per day for a specified duration.

In one example, the duration can be from a single day to a month.

In one example, symptoms of the condition can be reduced by at least about 10% since commencement of the treatment.

In one example, the composition can be in an oral dosage form.

In one example, the composition can further comprise a stabilizing carrier.

In one example, the stabilizing carrier can comprise deoxygenated water.

In one example, the stabilizing carrier can consist essentially of deoxygenated water.

In one example, the stabilizing carrier can further comprise one or more of: binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof.

In one example, the carrier can comprise a binder.

In one example, the therapeutically effective amount of rGSH can be from about 50 mg to about 1500 mg.

In one example, the method can further comprise co-administering an active agent to the subject.

In one example, the co-administration can be concomitant administration.

In one example, the active agent can be in the same or a different composition as the rGSH.

In one example, the active agent can comprise a member selected from the group consisting of: an antiviral, an antioxidant, an antitoxin agent, N-acetyl-cysteine, hydroxytyrosol (HXT), superoxide dismutase (SOD), catalase, an anti-infective agent, an antibiotic, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

In one example, the method can further comprise stimulating an immune response.

In one example, the method can further comprise administering the composition to the subject according to a dosage regimen.

In one example, the dosage regimen can comprise administering the composition to the subject at least once per day for a specified duration.

In one example, the dosage regimen can comprise administering the composition to the subject multiple times per day for a specified duration.

In one example, the duration can be from a single day to a month.

In one example, symptoms of the condition can be reduced by at least about 10% since commencement of the treatment.

In one example, the composition can be in an oral dosage form.

In one example, the composition can further comprise a stabilizing carrier.

In one example, the stabilizing carrier can comprise deoxygenated water.

In one example, the stabilizing carrier can consist essentially of deoxygenated water.

In one example, the stabilizing carrier can further comprise one or more of: binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof.

In one example, the carrier can comprise a binder.

In one example, the therapeutically effective amount of rGSH can be from about 50 mg to about 1500 mg.

In one example, the method can further comprise co-administering an active agent to the subject.

In one example, the co-administration can be concomitant administration.

In one example, the active agent can be in the same or a different composition as the rGSH.

In one example, the active agent can comprise a member selected from the group consisting of: an antiviral, an antioxidant, an antitoxin agent, N-acetyl-cysteine, hydroxytyrosol (HXT), superoxide dismutase (SOD), catalase, an anti-infective agent, an antibiotic, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

In one example, more than about 80% of the rGSH can remain in a reduced form upon administration to the subject.

In one example, more than about 50% of the rGSH can remain in a reduced form upon reaching a situs of action in the subject.

In one example, the condition can be selected from the group consisting of: acetaminophen overdose, drug-resistant bacterial infection, liver failure, HIV, herpetic encephalopathy, cystic fibrosis, narcotic overdose, macular degeneration, cataracts, glaucoma, peanut allergies, and combinations thereof.

In one example, the condition can be selected from the group consisting of: diabetic neuropathy, post-herpetic neuralgia (PHN), cold sore development, alcohol toxicity, paracetamol toxicity, wet macular degeneration, cataracts formation, corneal ulcers, teeth cavities, sunburns, laser burns, Epstein-Barr virus diseases, wounds, scarring, chronic renal failure (CRF), nephropathy, heavy-metal poisoning, chemotherapy-induced aphthous mouth ulcers, post tonsil surgery, insect bites, poison ivy reactions, anaphylaxis, necrobiosis lipoidica diabeticorum, pulmonary fibrosis, chronic obstructive pulmonary disease (COPD), optic neuritis (ON), Leber hereditary optic neuropathy (LHON)-like optic neuropathy (LLON), non-arteritic anterior ischemic optic neuropathy (NAION), sporadic bilateral optic neuropathy (SBON), and combinations thereof.

### EXAMPLES

The following examples are provided to promote a more clear understanding of certain embodiments of the present disclosure, and are in no way meant as a limitation thereon.

### Example 1—Topical Administration of rGSH to Treat Tardive Dyskinesia

### Summary:

Tardive Dyskinesia (TD) is a severe extrapyramidal side effect that can be caused by long term use of antipsychotics such as neuroleptic drugs. While antipsychotics are the most common drugs to cause TD there are many other medications that can cause this extrapyramidal side effect. TD is characterized by involuntary muscle movement ranging from slight tremors, grimacing, tongue movements, lip smacking, lip puckering, and pursing of the lips to involuntary movement of the limbs, torso, fingers and sometimes the entire body. The purpose of this study was to determine if reduced glutathione (rGSH) is a viable treatment of TD. In the study, rGSH was topically administered to an 86-year old subject that was diagnosed with TD of unknown etiology. After 10 weeks of treatment, her case of TD resolved completely which increased her quality of life.

### Methods:

Reduced glutathione was administered to the subject 5 times per day in a solution in the amount of about 18.25 mg per spray or about 91.25 mg per administration. The subject cleaned the area of application with a disinfectant wipe before topical application of the reduced glutathione. The topical rGSH was administered in 5 sprays of rGSH for a total dosage of about 91.25 mg. The administered rGSH dried for about 30 seconds before the subject was re-clothed. The facial movements and body movements of the subject were filmed once per week to analyze progression or loss of TD symptoms.

### Report:

An 86-year-old subject was presented for admission to hospice with a terminal diagnosis of tardive dyskinesia (TD) of unknown etiology. While many cases of TD result from anti-psychotic treatments, there have been studies linking other medications to TD ranging from anti-congestants to mood stabilizers. Tardive Dyskinesia had started in the subject a few months before and had rapidly progressed to dysphagia, significant weight loss, and aphasia.

At the initial visit, the subject was examined. The subject had involuntary facial and jaw movements or tics occurring about every 1-2 seconds. The subject's right arm also involuntarily flexed at about the same rate as her facial tics. The subject's constant movement caused difficulty in ambulation and the subject used assistance going up and down stairs. The subject was also in pain from the dyskinesia which was treated with 50 mg tramadol once per day at night. The subject had difficulty masticating, was almost completely dysphagic, and had lost significant weight since the onset of TD.

Topical rGSH was prescribed for 5 applications of 5 sprays each day to: (a) her right arm, (b) right jaw and masseter area, and (c) her spine. The total amount of administered rGSH was a total of 25 squirts per day (which is approximately 456 mg of rGSH via a Glutathione supplement) as applied by the nursing staff.

After 21 days of rGSH administration, the subject's symptoms visibly decreased. The subject's involuntary jaw movements in her masseter muscles declined from about every 1-2 seconds to about every 4-5 seconds. The subject's involuntary arm movements also decreased from about every 1-2 seconds to about 4-5 seconds. However, the subject still had constant pain from all of the involuntary movements. The amount of the pain was treated with 50 mg of tramadol once per day at night.

After 42 days of rGSH administration, the subject's symptoms decreased further. Facial and arm movements had further decreased from about every 4-5 seconds to about every 10 seconds. The subject's ambulation was better and the subject no longer used assistance for walking up and down stairs. However, the subject still had pain in her shoulder, back, and jaw although the pain had substantially decreased from the 21 day period.

After 70 days of rGSH administration, the subject was substantially free of symptoms. The subject no longer had involuntary facial or arm movement. The subject was capable of ambulating and eating without assistance. The subject was substantially free of pain with dramatic mood elevation.

### Discussion:

Tardive Dyskinesia (TD) is an involuntary movement disorder ranging from slight tremors to the movement of the whole body. These typically extrapyramidal side effects can be caused by long term treatment with antipsychotic drugs, such as neuroleptic drugs, that act as dopamine antagonists. TD can be associated with oxidative stress in the striatum region of the brain—related to the regulation and control of movement.

Glutathione is involved in removing free oxygen radicals within our cells. There can be an increased amount of oxidized glutathione (GSSG) in the cerebrospinal fluid and blood of subjects with TD. This may be caused by oxidation in the cells, especially in the striatum, or this may be caused by gene mutations for glutathione synthesis, re-cycling, and function such as glutathione S-transferase, GSTM1, GSTP, and the like. Some studies have stated that glutathione cannot pass the Blood Brain Barrier (BBB), but other studies have contradicted this statement.

Multiple drugs have been approved to treat TD (e.g., dopamine depleter or dopamine receptor antagonists, such as quetiapine and olanzapine) but the side effects outweigh the benefits of these different drugs. If this first intervention is not pertinent or ineffective, the clinician can prescribe an antikinetic therapeutic agent, such astetrabenazine or an antioxidant. Cholinergic drugs, anticholinergic drugs, benzodiazepine drugs, GABAergic drugs, or amantadine drugs may not be efficacious. Therapeutics (e.g. ECT and TMS) may not provide adequate treatment for TD. With high resistance and discomfort for the patient, a neurosurgical intervention can be undertaken. These treatments are limited; therefore, TD can be prevented by limiting the prescription and doses of antipsychotics, regularly evaluating their side effects, and informing the patient of TD's risk. Other medications, such as decongestants, can also produce neurotoxic byproducts leading to TD symptoms.

Since TD has been linked to oxidative stress, antioxidants have been used to treat TD, but the results have been mixed. Vitamin E, vitamin C, NAC, and glutathione have been tried without replication. Maintaining rGSH in a de-oxygenated aqueous solution reduced until the time and of the application reversed the TD. This result was unexpected because previous attempts with glutathione had failed.

A properly reduced and absorbable glutathione can reduce the level of oxidation subjects who have taken antipsychotic drugs. Research has shown that subjects taking commonly used antipsychotics for schizophrenia and other mental health issues are also more susceptible to TD due to multiple gene polymorphisms that can lead a residuum of the antipsychotics to become neurotoxic due to a lack of cellular glutathione production. Some of these polymorphisms include the dopamine receptor genes DRD2 and DRD3, the dopamine transporter DAT, GSTP1, GSTM1, and the manganese superoxide dismutase MnSOD gene. These nonfunctional genes may be linked to neurotoxin formation and dysfunctional removal.

Because of the increased oxidative stress and side effects due to antipsychotic drugs (e.g., increased dopamine production creating oxidative byproducts in the striata and spinal fluid) glutathione may not be replenished. This increase in oxidative byproducts can prevent removal of the neurotoxic metabolites and can increase the oxidation level in the brain compared to baseline. The exogenous reduced glutathione can be utilized by macrophages and dendritic cells to remove these neurotoxins which helps neurons recover and heal which can aid in the resolution of tardive dyskinesia.

### Example 2—Administration of rGSH to Treat Fibromyalgia

### Summary:

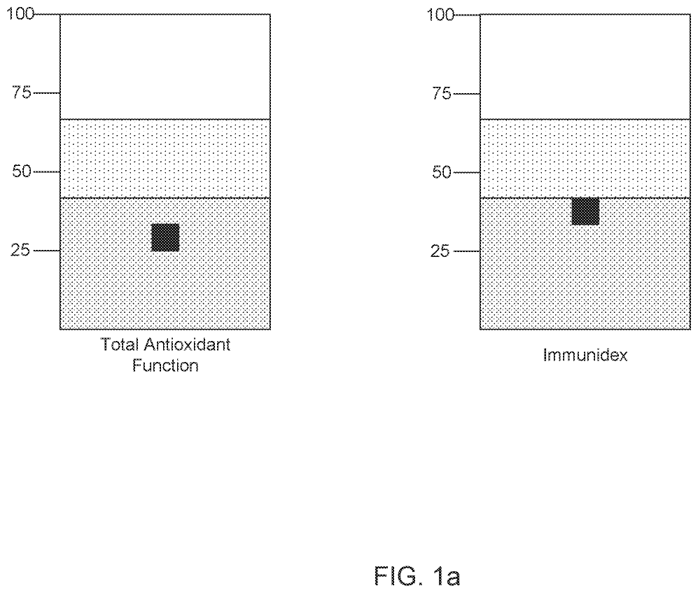

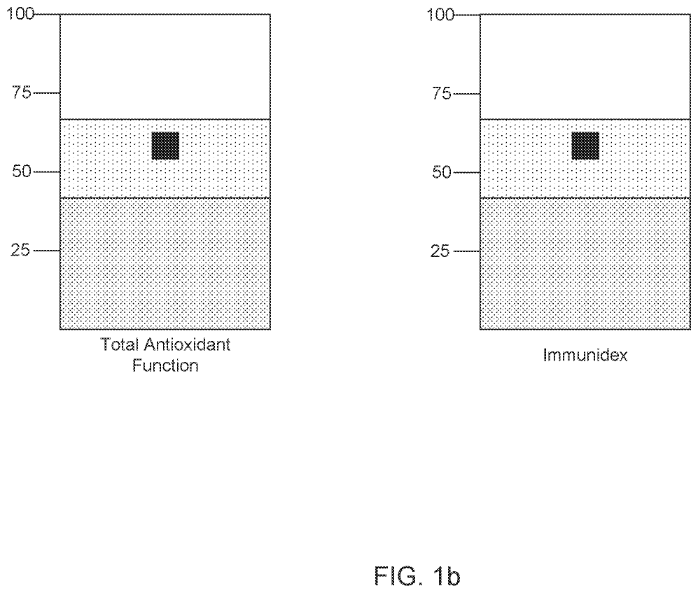

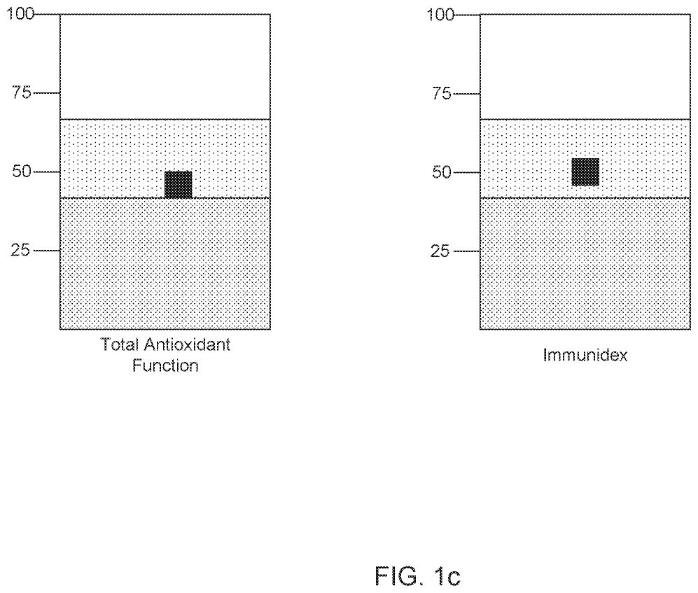

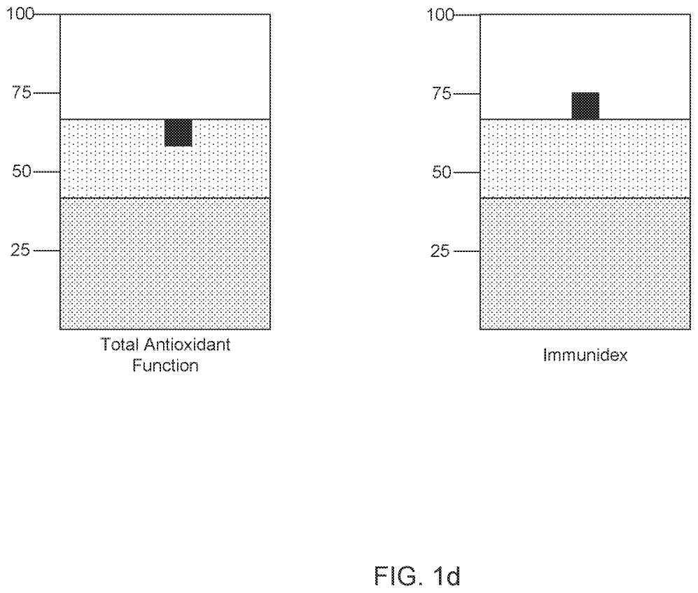

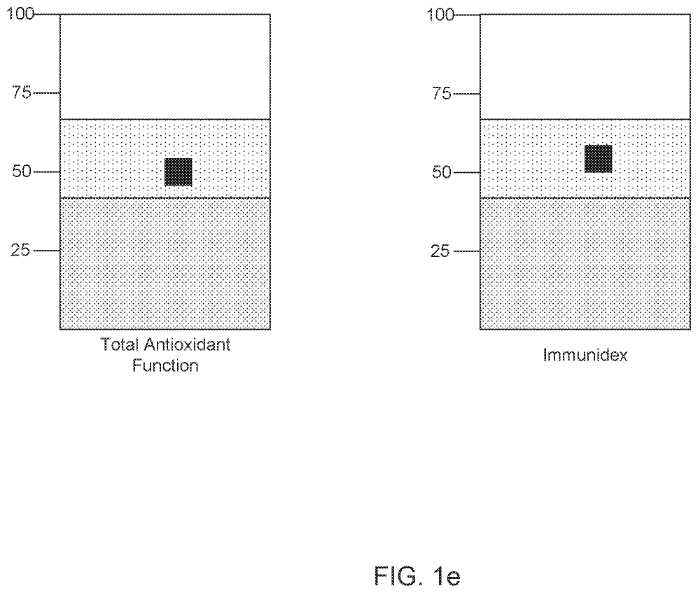

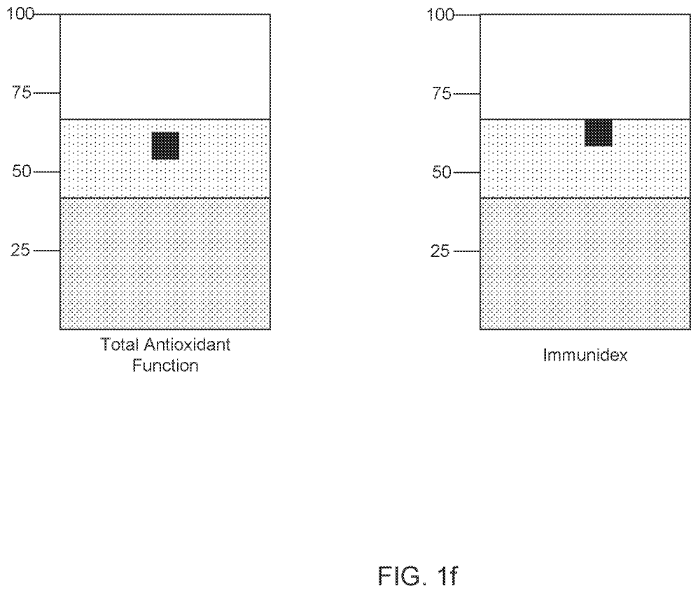

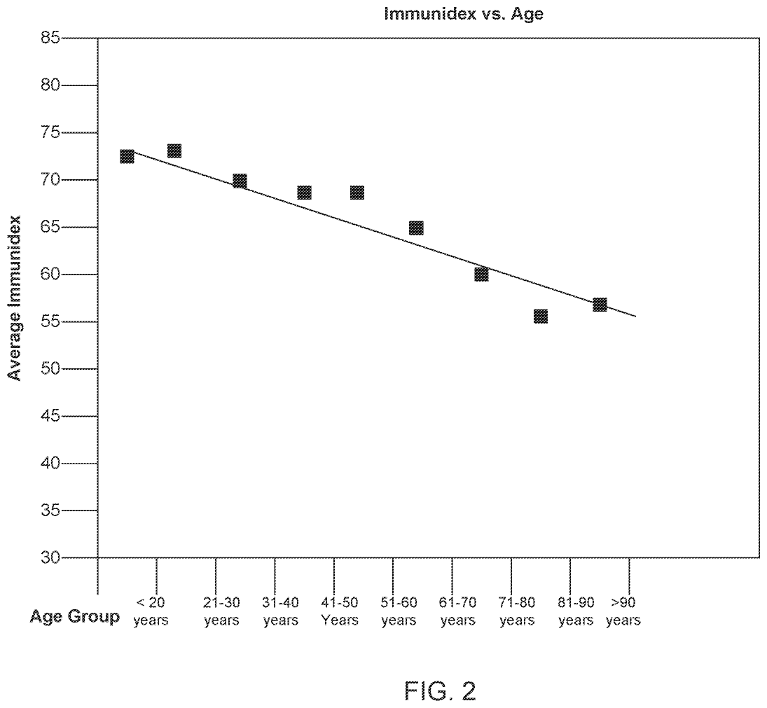

Fibromyalgia syndrome (FMS) subjects have higher levels of non-ceruloplasmin bound copper levels compared to baseline levels of non-ceruloplasmin bound copper levels. Non-ceruloplasmin bound copper levels were determined as from serum copper levels, serum zinc levels, and whole blood ceruloplasmin levels based on the NCC formula. Non-ceruloplasmin bound copper levels are related to a dysfunction in subjects relating to copper metallothionein (CMT). Functional CMT is used for cellular health. FMS was conjectured to be caused by copper toxicity related to molybdenum—a trace metal that can lead to CMT dysfunction. This error can result in an inability of the CMT to properly transport excessive copper from the extracellular space into the intracellular space, thus leading to excessive serum copper levels as determined by non-ceruloplasmin bound copper levels. It is conjectured that fibromyalgia symptoms of nerve pain, muscle tenderness and pain, joint arthralgias, fibrositis, and even increased seizures are due to CMT dysfunction. Treatment with reduced glutathione substantially reduced pain in the subjects. Reduced glutathione can increase the functionality of CMT resulting in mobilization of copper from the extracellular space to the intracellular space, and reducing oxidation, metal toxicity, and pain in the subjects. The 43 fibromyalgia subjects had substantial resolution of their FMS symptoms. See FIGS. 1a-1f and FIG. 2.

### Methods:

43 fibromyalgia subjects underwent dietary changes supplements including: (a) a low copper diet, (b) a support product containing molybdenum, boron, zinc chelate, pyridoxine, N-Acetyl L-Cysteine, L-serine and L-glutamine, and (c) rGSH.

### Report:

The aspects of fibromyalgia involving pain may be genetic, while the aspects of fibromyalgia involving fatigue may be caused by methylation errors. Chronic Fatigue Syndrome (CFS) associated with FMS can involve a genetic methylation error causing marked malabsorption at a cellular level of methyl-cobalamin, methyl-folate, choline, pantothenate. CFS with FMS can be treated with a support product containing molybdenum, boron, zinc chelate, pyridoxine, N-Acetyl L-Cysteine, L-serine and L-glutamine. rGSH is about one third by molecular weight of the Cu—Zn-Metallothionein and is important for the proper function of Cu—Zn-Metallothionein. Metallothionein attaches to and manipulates the copper molecule through the rGSH molecule. Without rGSH the Cu—Zn-Metallothionein complex does not function properly which can lead to a disparity in copper balance between extracellular copper levels and intracellular copper levels.

When methylation and deficiency therapy was undertaken, the symptoms of CFS resolved in a time period of from a few weeks to a few months. Although FMS may not resolve completely because of its genetic basis, the symptoms can be substantially resolved. For example, pain ratings can fall from about 8-10 to about 0-1, although occasional flares can occur with stress, weather changes, and dietary errors (e.g., accidentally eating a high copper food).

### Example 3—Topical Administration of rGSH to Treat Burns

### SUMMARY

An 86-year old female subject with partial and full thickness burn lesions on the neck and finger was treated with topical applications of rGSH 5-8 times per day over a 3-month period. Healing commenced immediately with minimal pain, minimal scarring, no infection, and minimal drainage. Therefore, the healing time was substantially reduced with and substantially free of pain. Topical application of reduced glutathione can treat subjects with mild to severe burn lesions.

### Methods:

The subject was treated with topical applications of rGSH. The subject was administered the rGSH 5-8 times a day over a 3-month period.

### Report:

An 86-yr-old female subject had accidentally burned herself on an outdoor grill. The subject was treated with rGSH at 5-8 times a day with topical application. The subject was also treated with Cephalexin 500 mg in case of infection.

On the day of the burn the subject applied rGSH for pain relief. The subject applied ice to her burn before applying the rGSH, possibly prolonging the healing. Within 30 minutes of topical application of rGSH, the subject reported the pain was subsiding and had nearly dissipated within an hour.

After 21 days of rGSH treatment, the subject's pain was tolerable but the subject's wound formed an eschar. Normal treatment would have removed the dead skin on day one, but the subject refused treatment in a burn unit. There was some pinkness around the area of the burn that was a concern for infection but none occurred.

After 35 days of rGSH treatment, the dead skin and eschar were gone and new skin has started to epithelialize. No infection had occurred and the subject was only in pain if she did not apply rGSH every 3-4 hours.

After 49 days of treatment, the wound had nearly healed with only hyperpigmented areas where the burn had occurred.

After 10 weeks after the burn, the subject was applying rGSH only rarely and no longer had any pain.

### Discussion:

Burns are a leading cause of trauma throughout the world. While many methods can treat burns, burns continue to be a high cause for morbidity and mortality throughout the world, especially for geriatric patients. Research has attempted to determine additional methods of treatment and cellular mechanisms and response. After a burn, there can be an increase in reactive oxygen species (ROS) in the cells and area surrounding the burn. Reduced glutathione can reduce the ROS, but the body's supply of rGSH can be quickly overwhelmed by the increased rate of ROS formation as shown by a higher rate of oxidized glutathione (GSSG) in the blood. The synthesis of new rGSH can be slowed after a major burn, and transport of rGSH from the liver can be slowed down as well. This reduced rate of rGSH synthesis may involve an intrinsic capacity for rGSH synthesis or may be caused by a lack of precursor amino acids needed for rGSH synthesis.

Because of increased ROS and low rGSH, cells surrounding the burned area can be quickly overwhelmed by the ROS, which can cause an inflammation cascade to the afflicted area. This mechanism can be used to fight infections that are common after burns, but ultimately slows down the healing process. Antioxidants have been used in the past to attempt to treat burns and reducing the amount of ROS, while decreasing the risk of infection and the healing time. A stable rGSH further reduce the increase in oxidation following a burn compared to other studies. Stable reduced glutathione may reduce ROS and inhibit the inflammation cascade to increase healing and reduce pain to the afflicted area that is caused by inflammation. Because rGSH has not shown this level of success in treating burns in previous studies, these results with stable rGSH were unexpected.

### Example 4—Topical Administration of rGSH to Treat Shingles

### Summary:

Over a 5-week period, 5 subjects with severe chronic shingles or Post-Herpetic Neuralgia (PHN), were treated with rGSH in a natural liposomal carrier. These subjects had an average age of approximately 73 years. Antivirals, narcotics, and other treatments (e.g., NSAIDs or tricyclic antidepressants) had failed to resolve their symptoms. Most subjects had pain self-ratings of 4-10 with an average of 7 on a scale of 1-10. The average length of break-out was about 12 months with one being as long as 2 years. Most subjects had lost hope and were severely depressed.

On the first visit all 5 subjects were treated with 3-4 sprays directly on the site with reduced glutathione in a liposomal carrier. All 5 subjects reported complete resolution of the pain within less than 34 minutes. With repeated application of reduced glutathione in a liposomal carrier about every 4-6 hours which was gradually decreased to once per day, (a) the pain associated with PHN did not return, (b) all skin lesions healed within 72 hours with minimal scarring, and (c) further break-outs ceased.

### Methods:

5 subjects having an average age of 77 had chronic shingles and severe post-herpetic neuralgia. The 5 subjects were treated over a five-week period of time with 3-4 sprays directly on the site with reduced glutathione in a liposomal carrier about every 4-6 hours which gradually decreased to once per day. Some of the subjects were on chronic doses of acyclovir, tricyclic antidepressants, and narcotics for pain without substantial ameliorative effect. Most the subjects at the time of the first visit reported significant depression and suicidal thoughts. Most subjects also reported giving up hope. All 5 subjects were asked to self-rate their pain at the time of admittance to therapy and during the study. The self-rated pain averaged a 7, but many subjects reported that when a break-out was occurring, the pain could spike to a 10. Videos and pictures were taken where and when appropriate with proper releases documented.

### Report:

Upon application of the spray of the reduced glutathione in a liposomal carrier, the pain resolved to zero (0) within an average of 34 minutes of application in all patients. Most subjects had complete resolution of symptoms within one minute or less. The shingles were resolved thereafter with minimal pain recurrences and minimal lesions. The pain was reported to have recurred when subjects waited more than 6 hours to re-apply the complexed glutathione on the first or second day of the study. Reports further indicated that there were no recurrences of any shingles outbreaks. Of interest, a few of the subjects reported wet macular degeneration which may be due to a systemic glutathione deficiency.

Subsequently, over a hundred patients and volunteers with shingles have been treated. In one case, a subject with shingles and PHN for about nine years had nearly complete resolution of symptoms within 34 minutes. Less than about 5% of subjects had nearly complete resolution over a more extended period ranging from 2-5 days.

### Discussion:

Chronic Shingles (along with PHN— Post-Herpetic Neuralgia) is a major public health concern. In one Italian study, PHN was still present after months of illness—370 of the 413 patients (89.6%) reported HZ-associated pain which was still present in 20.6% and 9.2% of patients after three and six months, respectively, despite many patients receiving recommended antiviral therapy. Herpes Zoster occurs in more than 500,000 people in the United States each year, during the lifetimes of as much as 20%-30% of the population, and in as many as 50% of those living until at least 85 years of age. The British Medical Council journal reported that “up to 20% of patients with HZ (herpes zoster) develop PHN, which is moderate-to-severe chronic pain persisting for months or years after the acute phase.” In 2001 the Mayo Clinic noted that, “the annual medical care cost of treating incident HZ cases in the United States, extrapolated from the results of this study in Olmsted County, is estimated at $1.1 billion.” PHN or post-herpetic neuralgia is one of the most common causes of suicide in patients with chronic pain over the age of 70 in the United States and Western Europe. In spite of treatments with antiviral medications, narcotics, and pain patches, pain is difficult to resolve and can cause heavy opioid use and addiction.

Topical glutathione has been used for shingles and other viral infections, but production and levels of rGSH decline as subjects age. Genetic errors such as GSTM1, GSTT1, and other genetic errors and polymorphisms can lead to inadequate amounts of reduced glutathione (rGSH) production. This decreased production of rGSH can decrease the reduction of oxidized glutathione (GSSG) to reduced glutathione (rGSH), which can increase toxic elements and cause dysfunctional metallothioneins.

Reduced glutathione is also used to provide immunity from viruses such as varicella-zoster, EBV, HIV, and other diseases. Glutathione therapy has been hindered by the lack of a stable reduced form that was easily applicable and truly functional. The stabilized, reduced, absorbable glutathione (rGSH) that is sterile and can be sprayed on topically has led to unexpected results in quickly resolving acute and long-standing shingles cases and PHN.

Stable rGSH can treat symptoms associated with chronic shingles and postherpetic neuralgia. rGSH can treat shingles by removing reactive oxygen species (ROS) within virally infected cells and directly inhibiting the late end stages of the herpes virus life cycle. rGSH can coordinate and activate various immune cells (e.g., macrophages) to process antigens to activate CD4+ T cells. These CD4+ T cells can activate CD8+T killer cells to produce a Th1 response to fight viruses. When endogenous rGSH is low in the body, then macrophages may not function properly, which can lead to a CD8+ T cell response of Th2 cells to fight bacteria. Proper rGSH levels that are provided exogenously to the immune system can correct the immunological response. Immune cells are enhanced in function and the rGSH can directly inhibit the late stage replication of HSV-1 in vitro. Therefore, stable rGSH has antiviral activity.

Postherpetic neuralgia is a complication associated with shingles that is characterized by pain or a burning sensation where the shingles rash occurred. This pain can be debilitating to the patient and can last weeks to years after the initial shingles infection has cleared out. This commonly leads to pain control with opioids and can lead to addiction. PHN can occur when a virus outbreak from nerves underneath the skin leads to permanent nerve damage. Stable rGSH has been shown to have many different neuroprotective benefits as well as helping repair damaged nerve cells. Stable rGSH can clear and remove any virally infected cells and repair damaged nerve cells during and after shingles and PHN.

### Example 5—Topical and Oral Administration of rGSH to Treat Chemotherapy-Induced Peripheral Neuropathy (CIPN)

### Summary:

A serious side effect of chemotherapy that results from taxane and platinum forms of chemotherapy is chemotherapy-induced peripheral neuropathy (CIPN). CIPN causes numbness and pain in the extremities due to nerve damage. Subjects were treated with topical and oral rGSH in a natural liposomal carrier. 4 subjects with CIPN were studied over a period of time ranging from a few months to 7 years. Each of the 4 cases saw substantial relief of symptoms.

### Methods:

Subjects were treated with topical and oral rGSH in a natural liposomal carrier after initial consultation. Subjects began treatment prior to their second visit. Follow-up consultations with subjects occurred weekly to monthly according to symptoms and needs. Levels of glutathione recommended varied as symptoms improved. 4 subjects participated in this study, and all had substantial resolution of their CIPN symptoms.

### Report:

Subjects were chemotherapy treated individuals with diagnosed CIPN. Subjects length of diagnosis for CIPN varied from a few months to seven (7) years. The subjects were not responsive to other treatments. The symptoms of CIPN was present in feet, legs, and hands of the subjects. All 4 subjects reported numbness and paresthesia to their knees, and none above their knees unless in their hands. At the beginning of treatment, each subject was experiencing CIPN symptoms including tingling, pain, loss of vibratory and positional sense, and numbness.

Subjects were not included in the study for (1) medical conditions that would create unnecessary hazards, or (2) if they were taking concurrently any agents to try to prevent or treat neuropathy, or (3) if they had active cancer undergoing treatment.

Procedures for measuring CIPN were based on interviews performed by a medical professional with subjects. The physician measured nerve sensitivity in some of the subjects using in-office medical instruments.

### Subject 5-A:

Day 1: Subject 5-A had breast cancer and had finished chemo treatment. Some of the subject's chemo drugs included cytoxan, adriamycin (red devil), and taxol. Taxol may have led to the development of CIPN. The subject had also gone through 25 rounds of radiation. CIPN development in the subject had started after cancer treatments affecting both hands and feet. The subject's finger pads had severe symptoms which made it difficult to type. Tingling and numbness went from the upper arm all the way down to the fingertips. The subject also had numbness and tingling on the bottom of feet. During the brush test, the subject reported that affected areas felt like gravel. During the sharp test, the subject reported pain up to the tops of the arms and up to the mid shins (i.e. 6 inches above the ankle).

Day 12: After 12 days of treatment, the CIPN symptoms had resolved to about 40% as reported by the subject. The sensation of symptoms changed in the subject and pain had moved to only the fingertips and nails. The affected area of her legs had moved to only the soles of the feet. The subject no longer reported pain in the shins. The subject was not applying rGSH topically as prescribed but had only took rGHS orally.

### Subject 5-B:

Subject 5-B had B cell lymphoma. During treatment the subject would spend 5 days in the hospital for chemotherapy. This included taking vintristen for 5 straight days using the dose adjusted epoc method. This treatment was done 6 times. The subject had CIPN for 3 years affecting the hands (from the wrist down), and legs (from the knees down) before starting treatment with rGSH.

Day 7: One week after initially taking rGSH, the subject had shown improved symptoms. Feeling had started to return to the hands and feet, but pain was still occurring.

Day 14: Symptoms had continued to improve with the subject reporting no more pain in the fingers. However, the subject reported that feeling had not been fully restored to the fingertips. There subject still reported pain from the knees down, although the level of pain had decreased. The subject had not been applying rGSH to the fingertips, but was still applying orally between 5 pumps at 3 times a day and 4 pumps at 5 times a day to legs and feet.

Day 28: Subject reported that the symptoms were about the same, but had run out of rGSH without receiving more rGSH.

Day 42: The subject reported 50% improvement of symptoms since using rGSH. The subject's feet were less inflamed, and the subject was able to wear shoes without discomfort. The subject reported no longer feeling any pain in her calves and only had pain in the soles of her feet.

Day 56: No major change in symptoms reported by the subject. The subject reported sleeping through the night without waking up in pain and was able to stand up more. The subject reported no pain in the legs unless stressed, though the subject still reported constant pain in the feet.

Day 84: Subject reported 70% resolution of CIPN symptoms, but the bottom of the feet still had some numbness and tingling.

Day 112: Subject reported 70% resolution of CIPN symptoms, with no major change of symptoms.

Day 245: Subject had stopped taking rGSH for a few months, and was still reporting symptoms at 70% resolution. The subject had no regression of symptoms, but no progress since stopping rGSH.

### Patient 5-C:

This subject had CIPN for two years, and had platinum-based chemotherapy drugs during treatment.

Day 49: Within 3 days, the subject reported a major resolution of symptoms. The subject's hands were no longer numb or in pain, and the subject had increased feeling in fingers. The subject's legs took longer to resolve but the majority of pain only affected the soles of the feet.

Day 63: By this point the subject reported pain and numbness every few days. The subject no longer felt pain in the feet and was able to walk normally again, with some problems when the feet were numb. The subject was still taking rGSH orally 2 to 3 times a day, and only applying rGSH topically to affected areas about twice a day.

### Patient 5-D:

Subject has multiple myeloma and was prescribed revlimid. The patient was on revlimid for a year. The subject developed CIPN during that year and had CIPN for about 11 years. The subject reported minor CIPN symptoms that only affected the balls of the feet and toes. The subject did not report any major discomfort, but described the feeling of walking on gravel all the time unless they were walking barefoot on hardwood floor. The subject reported that walking barefoot on hardwood floor was painful. The subject also reported pain when driving. The subject reported it felt like sharp rocks were poking the right big toe while driving, and reported more problems in the right foot in comparison to the left. CIPN did not affect the subject's balance, and the subject reported that the symptoms were mostly annoying rather than painful.

Day 1: Subject topically applied rGSH 3-5 times a day to feet. Subject took rGSH twice a day orally.

Day 10: Subject reported decreased symptoms within a few days with less pain in the big toe, but the pain in the big toe returned.

Day 31: Subject reported substantial decrease in symptoms. Subject reported sharp pain on the big toe was no longer as painful, but still felt like gravel under the feet.

Day 38: Subject reported no discomfort in feet, even when driving. Subject reported that the sharpness of pain in the big toe was almost gone. The subject reported feeling about 70% better since starting treatment.

Day 52: Subject reported walking barefoot on hardwood floors without any discomfort or pain. Subject reported an 80% decrease in symptoms.

Day 68: Subject reported stagnation of symptoms and felt more comfortable walking around barefoot.

Day 94: Subject reported no major change in symptoms. Subject reported some feeling of small gravel under toes on occasion, but without any more pain.

### Discussion:

CIPN is caused by multiple substance groups including platinum-based antienoplasts agents, vinca alkaloids, epothilones (ixabepilone), taxanes, proteasome inhibitors (bortezomib) and immunomodulatory drugs (thalidomide). It has been estimated that there are 100,000-300,000 new cases of CIPN every year, and an estimated number of 10 million Americans are currently afflicted with CIPN. The platinum-based drugs can cause about 70% of patients to develop CIPN after treatment. These chemotherapy drugs cause damage to nerve cells by killing them directly or causing them to be ineffective. This can lead to CIPN symptoms such as numbness of distal appendages, shooting pain, and difficulty walking.

With few treatments for CIPN without side effects, reduced glutathione was tested to relieve CIPN symptoms. Many platinum-based drugs attack the mitochondria of rapidly dividing cells which can increase production of reactive oxygen species and alter mitochondria function to induce apoptosis. This can kill cancer cells but also kill or damage the subjects own neurons and lead to neuropathy.

Because many reactive oxygen species are created during the respiration processes of the electron transport chain, stable rGSH may: (a) clear away these ROS and increase nerve function and neuron conduction, and (b) remove remaining chemo drugs (e.g., platinum-based neurotoxins) persisting in dorsal root ganglia that might be causing CIPN effects. By using these mechanisms, rGSH can restore nerve cells and reduce CIPN symptoms. Previous studies were unsuccessful in treating CIPN with rGSH. The positive findings from this study confirm the benefits of stable rGSH in treating CIPN.

### Example 6—Topical and Oral Administration of rGSH to Treat Peripheral Neuropathy

### Summary:

Diabetic Peripheral Neuropathy (DN) is characterized by a numbness of extremities, hands, and feet leading to tingling and pain in affected areas. DN is a secondary effect resulting from oxidative stress occurring from hyperglycemia in patients with diabetes. Common side effects of hyperglycemia lead to high amounts of reactive oxygen species (ROS) in the body that can cause neurological damage as well as cause cellular death through apoptosis. Reduced Glutathione (rGSH) is an antioxidant involved in removing ROS. Subjects with DN are unable to synthesize adequate levels of rGSH to neutralize the ROS that produce associated side effects. Exogenously administration of stable rGSH topically to the affected areas of the subject over the course of 13 weeks resolved the subject's 9 year case of DN.

### Methods:

Neurological examination of the subject showed: (i) absent bilateral Achilles tendon deep tendon reflexes, (ii) absent light touch (horse hair), (iii) absent ability to determine blinded digital or ankle positional sense, (iv) absent vibration sensation up to the level of the plateau, and (v) absent to pain (pin prick) to the level of the distal portion of the patella. The subject's muscle strength was weak, and the subject's ambulation was poor without assistance. The subject's heart rate and blood pressure were both normal. The subject's neuropathic distribution was clear cut to the edge of the distal patella bilaterally, and to the elbows on the upper extremities.

The subject started the treatment with topical rGSH 4-8 times per day, and the subject also started treatment with oral rGSH. The subject applied the rGSH topically to his feet and legs and rubbed it in using both hands. The subject's hands also had neuropathy. The subject attended weekly follow-up visits that were charted and video recorded. The subject was consistent in his topical therapy using the glutathione and additionally orally ingested approximately 550 mg (15 doses) of the VARS™ Glutathione every day.

### Report:

The subject is a 58-year-old man with a ten-year history of impaired glucose intolerance along with subsequent diabetic neuropathy with absence of sensation in his feet to just below his knees. The subject also has absence of sensation in his hands, but the lack of sensation is more severe in his feet and lower legs. He was forced to walk with a cane and had a halting gait consistent with his neuropathy. He was single, occasionally took testosterone cypionate injections, drank no alcohol, and never smoked. His neuropathy made his continued employment as a newspaper ad man difficult due to his awkward ambulation. He was on no pain meds for his neuropathy.

9 years before starting treatment, the subject had noticed a burning and tingling in his feet. It had become quite uncomfortable for a few years and thereafter had him on pain medications. About 6 years before starting treatment, his feet and hands had become numb. He had found no therapy that could help. It was very difficult for him to exercise and he ambulated only with the assistance of a cane. He worried about falling because he could not feel his lower legs or feet.

A review of the subject's medical records revealed fasting and premeal glucose levels generally less than about 150 mg/dL and postprandial levels greater than about 180 mg/dL. His most recent HBA1C was 10.0% (outside the normal range of 4.0-6.0%) showing poor control of his diabetes. A complete blood count, lipid panel, liver screening, renal profile, and PSA test were all normal.

The subject reported a low libido and neuropathy. The patient had commenced treatment with a modest dose of injectable testosterone to improve his insulin sensitivity, lower muscle strength to aid in ambulation, and improve his neuropathy. The injectable testosterone treatment did not have a substantial effect on the subject's issues.

Because seven other subjects had reported complete resolution of their diabetic neuropathy when treated with stable rGSH, the subject was treated with oral and topical rGSH. His neuropathy rapidly improved based on weekly examination, with a complete resolution in his hands in the first few weeks, then improving to mid-shin, then lower shin, then ankles, then midfoot, then to the toes, and then finally complete resolution. He had noticeable and complete resolution in his absence of light touch, pin-prick, vibratory sense, and positional sense but had no DTR's still in any of his extremities. Ambulation had noticeably improved and was stronger and more assured in his foot placement and stride.

The subject's neuropathy resolved substantially after 13 weeks total of the therapy. His HBA1C improved modestly to 10.1%. No effect was noted on his renal or liver functions. A small non-itchy raised scaled rash on his left anterior mid shin was noted and he was referred to a dermatologist.

The subject was advised to use the topical rGSH sparingly 1-2 times per day to prevent a recurrence of the neuropathy. He was also advised to start walking more in order to build up his leg strength and gait.

### Discussion:

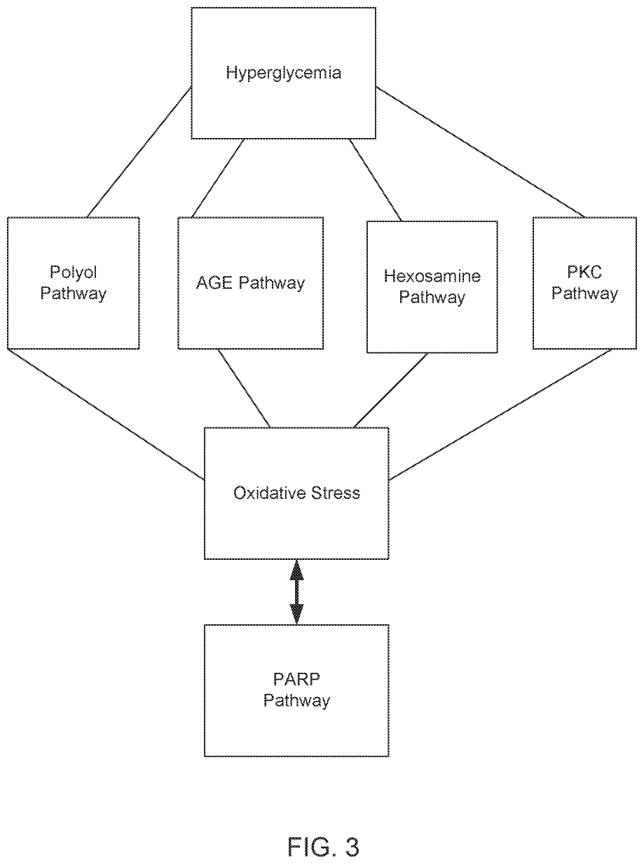

Hyperglycemia is a major side effect of diabetes. The increased glucose creates a massive oxidative strain in the cells of the body by the autoxidation of glucose and the glycosylation of other proteins. The increase in reactive oxygen species (ROS) can damage cellular macromolecules (e.g., carbohydrates, proteins, DNA and lipids) which can lead to loss of function of structures and genes. A stress response of the cell can activate certain genes that are proinflammatory, such TNF-alpha and IL-1beta, leading to further complications of diabetes. See FIG. 3.

High ROS can activate the Bax-caspase pathway involved in cell apoptosis, causing the mitochondrial membrane leak cytochrome c into the cytoplasm and activating the caspases that lead to apoptosis. The destruction of cells like neurons caused by ROS and inflammatory responses in the body can result in neuropathy in diabetic patients. Some treatments attempt to block oxidative pathways such as the polyol pathway, AGE pathway, hexosamine pathway, and the PKC pathway. While direct inhibition of these pathways can relieve symptoms of neuropathy, this approach does not decrease the high rate of ROS in cells.

Because antioxidants have been used to treat DN without positive results, the results obtained in this study are unexpected. Therefore, treating a cause of DN (i.e. an increase in ROS) can provide an effective treatment. Previous attempts did not use adequate amounts of rGSH because of instability with the reduced form of glutathione.

It is understood that the above-described various types of compositions, dosage forms and/or modes of applications are only illustrative of preferred embodiments of the present disclosure. Numerous modifications and alternative arrangements may be devised by those skilled in the art without departing from the spirit and scope of the present disclosure and the appended claims are intended to cover such modifications and arrangements. Thus, while the present disclosure has been described above with particularity and detail in connection with what is presently deemed to be the most practical and preferred embodiments of the disclosure, it will be apparent to those of ordinary skill in the art that variations including, but not limited to, variations in size, materials, shape, form, function and manner of operation, assembly and use may be made without departing from the principles and concepts set forth herein.

1. A method of treating a subject having a condition responsive to treatment with reduced glutathione (rGSH), comprising:
administering a therapeutically effective amount of a substantially α-D-glucopyranoside-free stabilized rGSH composition to the subject.

2. The method of claim 1, wherein the condition is a skin condition.

3. The method of claim 2, wherein the skin condition is selected from the group consisting of: psoriasis, eczema, acne, hives, warts, cold sores, candidiasis, athlete's foot, wounds, mouth wounds, mouth pain, burns, sunburns, dry skin, wrinkles, blisters, actinic keratosis, rosacea, carbuncles, cellulitis, contact dermatitis, and keratosis pilaris.

4. The method of claim 3, wherein the skin condition is one of a burn, a wound, or eczema, and the rGSH is present in the composition in an amount of from about 50 mg to about 1500 mg.

5-6. (canceled)

7. The method of claim 4, further comprising administering the composition to the subject according to a dosage regimen.

8-10. (canceled)

11. The method of claim 7, wherein symptoms of the condition are reduced by at least about 10% since commencement of the treatment.

12. The method of claim 2, wherein the composition is in a topical dosage form.

13. The method of claim 12, wherein the composition further comprises a stabilizing carrier.

14. The method of claim 13, wherein the stabilizing carrier comprises deoxygenated water.

15. The method of claim 13, wherein the stabilizing carrier consists essentially of deoxygenated water.

16. The method of claim 14, wherein the stabilizing carrier further comprises a solution, a suspension, an emulsion, a gel, a hydrogel, a thermo-responsive gel, a cream, a paste, or an ointment.

17-18. (canceled)

19. The method of claim 2, further comprising co-administering an active agent to the subject.

20. The method of claim 19, wherein the co-administration is concomitant administration.

21. The method of claim 20, wherein the active agent is in the same or a different composition as the rGSH.

22. The method of claim 19, wherein the active agent is a member selected from the group consisting of: an antioxidant, an antibiotic, an antiviral, an antitoxin agent, hydroxytyrosol (HXT), superoxide dismutase (SOD), catalase, an anti-infective agent, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

23. The method of claim 20, wherein the condition is a burn and the active agent is an antibiotic.

24. The method of claim 20, wherein the condition is a wound and the active agent is an antibiotic.

25. The method of claim 20, wherein the condition is eczema and the active agent is an antioxidant.

26-131. (canceled)

132. The method of claim 1, wherein more than about 80% of the rGSH remains in a reduced form upon administration to the subject.

133. The method of claim 1, wherein more than about 50% of the rGSH remains in a reduced form upon reaching a situs of action in the subject.

134-135. (canceled)
